(19) United States
(12) Patent Application Publication
(10) Pub. No.: US 2025/0387056 A1
(43) Pub. Date: Dec. 25, 2025

(54) Arterial Blood Gas Syringe

(71) Applicant: Becton, Dickinson and Company, Franklin Lakes, NJ (US)

(72) Inventors: Anthony V. Torris, Mercer Island, WA (US); William Thomas Kelly, White Plains, NY (US); Richard A. Cronenberg, Mahwah, NJ (US); Carlos Eduardo Sanchez, Butler, NJ (US); Michael Vincent Quinn, East Hanover, NJ (US); David Robert Schiff, Highland Park, NJ (US); Donald Fraser VanRoyen, Philadelphia, PA (US); Brian Weingarth, Philadelphia, PA (US); Ben Bernstein, Philadelphia, PA (US)

(21) Appl. No.: 18/854,610

(22) PCT Filed: Mar. 31, 2023
(86) PCT No.: PCT/US2023/017108

§ 371 Date: Oct. 7, 2024

(51) Int. Cl. A61B 5/153; A61B 5/15

(52) U.S. Cl. CPC A61B 5/153; A61B 5/15003; A61B 5/150213; A61B 5/150221; A61B 5/150519; A61B 5/150656

(57) ABSTRACT

The arterial blood module including an arterial blood collection element defining a collection chamber, and an arterial blood module removably connectable to a portion of the arterial blood collection element. The arterial blood module includes a hub body, a needle extending from the hub body, and a safety shield engaged with a portion of the hub body and transitionable from a first shield position in which a portion of the needle is exposed to a second shield position in which the needle is shielded by a portion of the safety shield. The arterial blood module also includes a venting chamber, wherein the venting is in fluid communication with the hub body to vent air bubbles from a collected blood sample within the collection chamber of the arterial blood collection element.

### CROSS-REFERENCE TO RELATED APPLICATION

The present application claims priority to U.S. Provisional Application Ser. No. 63/328,960, entitled “Arterial Blood Gas Syringe”, filed Apr. 8, 2022, the entire disclosure of which is hereby incorporated by reference in its' entirety.

### BACKGROUND OF THE INVENTION

### Field of the Disclosure

The present disclosure relates generally to arterial blood collection systems.

More particularly, the present disclosure relates to arterial blood collection systems including arterial blood collection elements suitable for radial artery stick and arterial blood modules.

### Description of the Related Art

Arterial blood collection syringes are used for withdrawing and collecting arterial blood samples from the body of a patient. Once the blood sample is collected, it is subjected to diagnostic analysis for primarily blood gases and often also electrolytes, metabolites, and other elements that are indicative of a condition of a patient. Various types of syringes have been devised for collecting arterial blood samples, which mainly comprise elements from a hypodermic syringe, i.e., a plastic or glass syringe barrel, a sealing elastomeric stopper with or without air vent, and a plunger rod.

Conventional arterial blood collection syringes typically use conventional hypodermic needles with a safety shield that need to be snapped or slid over the needle after a blood collection procedure. Such safety guards are often in the line of sight during a blood collection procedure thereby obscuring a physician's view during this delicate procedure.

Conventional arterial blood collection syringes also have a separate vent cap that requires the needle to be removed before the cap is attached to the syringe to expel trapped air bubbles from a collected sample.

Conventional arterial blood collection syringes that include anticoagulant typically are loaded with the anticoagulant inside the syringe, thus, requiring a user to roll or shake a collected sample to ensure thorough mixing with the anticoagulant.

Arterial blood modules which are removably connectable to an arterial blood collection element have been contemplated. For example, U.S. application Ser. No. 17/272,767, published as U.S. Application Publication No. 2021/0315498 and incorporated herein by reference in its entirety, discloses arterial blood modules removably connectable to an arterial blood collection element.

### SUMMARY OF THE INVENTION

The present disclosure provides an arterial blood module removably connectable to an arterial blood collection element. The arterial blood module includes a housing, a needle, a cap removably securable over the needle, a mixing chamber, and a safety shield engaged with a portion of the housing and transitionable from a first shield position in which a portion of the needle is exposed to a second shield position in which the needle is shielded by a portion of the safety shield. In one embodiment, the present disclosure includes an arterial blood collection system that includes an arterial blood collection element defining a collection chamber and an arterial blood module removably connectable to a portion of the arterial blood collection element. Additional and/or alternatively, an arterial blood module in accordance with the present disclosure may be provided without a mixing chamber.

In an exemplary embodiment, the arterial blood collection system of the present disclosure provides a novel blood collection device for collecting of arterial blood samples using a radial stick technique. In other embodiments, the arterial blood collection system in accordance with aspects of the present disclosure may be used for collecting arterial blood samples from the brachial artery. The system of the present disclosure provides an efficient system that streamlines and reduces the number of workflow steps and enables singlehanded device operation which allows for more efficient Arterial Blood Gas (ABG) collection procedures. The arterial blood module of the present disclosure includes ergonomic touch points, push button safety shield, automatic anticoagulant mixing and integrated vent cap for expelling air bubbles after a collection procedure, if necessary.

In accordance with an embodiment of the present invention, an arterial blood collection system includes an arterial blood collection element defining a collection chamber; and an arterial blood module removably connectable to a portion of the arterial blood collection element, the arterial blood module comprising: a housing having a first end and a second end; a needle extending from the first end; a cap removably securable over the needle; a mixing chamber adjacent the second end; and a safety shield engaged with a portion of the housing and transitionable from a first shield position in which a portion of the needle is exposed to a second shield position in which the needle is shielded by a portion of the safety shield.

In one configuration, the safety shield comprises a shield assembly; and an actuator transitionable from a first actuator position in which the actuator engages a portion of the shield assembly to lock the shield assembly in the first shield position to a second actuator position in which the actuator releases the shield assembly and the safety shield automatically moves to the second shield position. In another configuration, the actuator comprises a push button. In yet another configuration, the shield assembly comprises telescoping shields. In one configuration, the shield assembly comprises a fixed outer shield; a middle movable shield in communication with the fixed outer shield, wherein the middle movable shield moves relative to the fixed outer shield; and an inner movable shield in communication with the middle movable shield, wherein the inner movable shield moves relative to the middle movable shield. In another configuration, with the safety shield in the first shield position, the inner movable shield is nested inside the middle movable shield, and the middle movable shield is nested inside the fixed outer shield. In yet another configuration, with the safety shield in the second shield position, the inner movable shield extends from the middle movable shield, and the middle movable shield extends from the fixed outer shield. In one configuration, the housing of the arterial blood module defines a flow channel from the first end to the second end. In another configuration, the arterial blood module further comprises a sample stabilizer disposed within the flow channel between the first end of the housing and the mixing chamber. In yet another configuration, the arterial blood collection system includes a material including pores disposed within the flow channel between the first end of the housing and the mixing chamber; and a dry anticoagulant powder within the pores of the material. In one configuration, a blood sample dissolves and mixes with the dry anticoagulant powder while passing through the material. In another configuration, the material is an open cell foam. In yet another configuration, the sample stabilizer is the dry anticoagulant powder. In one configuration, the housing of the arterial blood module defines a vent chamber. In another configuration, the arterial blood module further comprises a venting plug that allows air to pass therethrough and prevents a blood sample from passing therethrough, wherein a portion of the venting plug is in communication with the vent chamber. In yet another configuration, the arterial blood module further comprises a first valve, and wherein, with the arterial blood module connected to the arterial blood collection element, the blood sample enters the collection chamber of the arterial blood collection element via the needle and the first valve. In one configuration, the first valve allows the blood sample to pass from the arterial blood module to the collection chamber of the arterial blood collection element. In another configuration, the first valve blocks the blood sample from passing from the collection chamber of the arterial blood collection element back to the arterial blood module. In yet another configuration, the arterial blood module further comprises a second valve, and wherein, with the arterial blood module connected to the arterial blood collection element, air contained in the collection chamber of the arterial blood collection element and a portion of the blood sample enter the vent chamber via the second valve. In one configuration, the air travels out of the arterial blood module via the venting plug. In another configuration, the arterial blood module includes a first finger grip portion. In yet another configuration, the arterial blood module includes a second finger grip portion, wherein the first finger grip portion is opposite the second finger grip portion. In one configuration, the needle comprises thin wall needle technology. In another configuration, the arterial blood collection element includes a plunger rod assembly including a stopper and a plunger rod.

In accordance with another embodiment of the present invention, an arterial blood module includes a housing having a first end and a second end; a needle extending from the first end; a cap removably securable over the needle; a mixing chamber adjacent the second end; and a safety shield engaged with a portion of the housing and transitionable from a first shield position in which a portion of the needle is exposed to a second shield position in which the needle is shielded by a portion of the safety shield.

In one configuration, the arterial blood module is removably connectable to a portion of an arterial blood collection element. In another configuration, the arterial blood collection element defines a collection chamber.

In accordance with another aspect of the present disclosure, an arterial blood collection system is disclosed including an arterial blood collection element defining a collection chamber, and an arterial blood module removably connectable to a portion of the arterial blood collection element. The arterial blood module may include a hub body, a needle extending from the hub body, and a safety shield engaged with a portion of the hub body and transitionable from a first shield position in which a portion of the needle is exposed to a second shield position in which the needle is shielded by a portion of the safety shield. The arterial blood module may also include a venting chamber, wherein the venting chamber is in fluid communication with the hub body to vent air bubbles from a collected blood sample within the collection chamber.

In some embodiments, the arterial blood module further includes an actuator transitionable from a first actuator position to a second actuator position, wherein the second actuator position releases the safety shield to the second shield position.

In some embodiments, the actuator includes a push button.

In some embodiments, the safety shield includes telescoping shields.

In some embodiments, the venting chamber is formed within the actuator.

In some embodiments, the hub body of the arterial blood module defines the venting chamber.

In some embodiments, the arterial blood module further includes a venting plug that allows air to pass therethrough and prevents a blood sample from passing therethrough, wherein a portion of the venting plug is in communication with the venting chamber.

In some embodiments, the needle and safety shield are selectively removable from the hub body.

In some embodiments, the needle and safety shield are removable from the hub body only when the safety shield is in the second shield position.

In some embodiments, the actuator includes an actuator lever and stopcock valve.

In some embodiments, the arterial blood collection module further includes an eccentric needle carrier, the eccentric needle carrier having a needle carrying portion coupled to and in fluid communication with the needle, a venting chamber portion, and a rotatable lever portion.

In some embodiments, the arterial blood collection module further includes an insert positioned at a distal end of an interior portion of the safety shield to seal an opening in the safety shield when in the second shield position in which the needle is shielded by a portion of the safety shield.

In accordance with another aspect of the present disclosure, an arterial blood module is disclosed, the arterial blood module including a hub body, a needle extending from the hub body, a safety shield engaged with a portion of the hub body and transitionable from a first shield position in which a portion of the needle is exposed to a second shield position in which the needle is shielded by a portion of the safety shield, and a venting chamber, wherein the venting chamber is in fluid communication with the hub body to vent air bubbles from a collected blood sample within the collection chamber.

In some embodiments, the arterial blood module is removably connectable to a portion of an arterial blood collection element.

In some embodiments, the arterial blood module is connectable to a portion of the arterial blood collection element by way of a threaded luer connection.

In some embodiments, the arterial blood module further includes an actuator transitionable from a first actuator position to a second actuator position, wherein the second actuator position releases the safety shield to the second shield position.

In some embodiments, the venting chamber is formed within the actuator.

In some embodiments, the actuator includes a push button.

In some embodiments, the actuator includes an actuator lever and stopcock valve.

In some embodiments, the hub body of the arterial blood module defines the venting chamber.

In some embodiments, the arterial blood module further includes a venting plug that allows air to pass therethrough and prevents a blood sample from passing therethrough, wherein a portion of the venting plug is in communication with the venting chamber.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of an arterial blood collection system in accordance with an embodiment of the present invention.

FIG. 2 is a perspective view of an arterial blood module with a cap secured over a needle in accordance with an embodiment of the present invention.

FIG. 3 is a perspective view of an arterial blood module with a cap removed from a needle in accordance with an embodiment of the present invention.

FIG. 4 is a perspective view of an arterial blood collection system with air bubbles being expelled from the system in accordance with an embodiment of the present invention.

FIG. 5 is a perspective view of a first step of using a system of the present disclosure in accordance with an embodiment of the present invention.

FIG. 6 is a perspective view of a second step of using a system of the present disclosure in accordance with an embodiment of the present invention.

FIG. 7 is a perspective view of a third step of using a system of the present disclosure in accordance with an embodiment of the present invention.

FIG. 8 is a perspective view of a fourth step of using a system of the present disclosure in accordance with an embodiment of the present invention.

FIG. 9 is a perspective view of a fifth step of using a system of the present disclosure in accordance with an embodiment of the present invention.

FIG. 10 is a first schematic view of an arterial blood collection system in accordance with an embodiment of the present invention.

FIG. 11 is a second schematic view of an arterial blood collection system in accordance with an embodiment of the present invention.

FIG. 12 is a third schematic view of an arterial blood collection system in accordance with an embodiment of the present invention.

FIG. 13 is a cross-sectional view of a portion of an arterial blood module with a safety shield in a first shield position in accordance with an embodiment of the present invention.

FIG. 14 is a cross-sectional view of a portion of an arterial blood module with a safety shield in a second shield position in accordance with an embodiment of the present invention.

FIG. 15 is a cross-sectional view of a portion of an arterial blood module with a safety shield in a first shield position in accordance with another embodiment of the present invention.

FIG. 16 is a cross-sectional view of a portion of an arterial blood module with a safety shield in a second shield position in accordance with another embodiment of the present invention.

FIG. 17 is a cross-sectional view of a portion of an arterial blood module with a safety shield in a first shield position in accordance with another embodiment of the present invention.

FIG. 18 is a cross-sectional view of a portion of an arterial blood module with a safety shield in a second shield position in accordance with another embodiment of the present invention.

FIG. 19 is a perspective view of an open cell foam material in accordance with an embodiment of the present invention.

FIG. 20 is a microscopic view of the microstructure of an open cell foam material having a dry anticoagulant powder distributed throughout its microstructure in accordance with an embodiment of the present invention.

FIG. 21 is a perspective view of an arterial blood module in accordance with another aspect of the present invention.

FIG. 22 is a cross-sectional view of the arterial blood module of FIG. 21 in a first configuration.

FIG. 23 is a cross-sectional view of the arterial blood module of FIG. 21 in a second configuration.

FIG. 24 is a cross-sectional view of the arterial blood module of FIG. 21 in a third configuration.

FIG. 25 is a perspective view of an arterial blood module in accordance with another aspect of the present invention.

FIG. 26 is a cross-sectional view of the arterial blood module of FIG. 25 in a first configuration.

FIG. 27 is a cross-sectional view of the arterial blood module of FIG. 25 in a second configuration.

FIG. 28 is a cross-sectional view of an arterial blood module in accordance with another aspect of the present invention in a first configuration.

FIG. 29 is a cross-sectional view of the arterial blood module of FIG. 28 in a second configuration.

FIG. 30 is a perspective view of an arterial blood module for an arterial blood gas syringe in accordance with another aspect of the present invention.

FIG. 31 is a cross-sectional view of the arterial blood module of FIG. 30 in a first configuration.

FIG. 32 is another cross-sectional view of the arterial blood module of FIG. 30 in the first configuration.

FIG. 33 is a cross-sectional view of the arterial blood module of FIG. 30 in a second configuration.

FIG. 34 is a cross-sectional view of the arterial blood module of FIG. 30 in a third configuration.

FIG. 35 is a cross-sectional view of the arterial blood module of FIG. 30 in a fourth configuration.

FIG. 36 is a cross-sectional view of the arterial blood module of FIG. 30 in a fifth configuration.

FIG. 37 is a perspective view of an arterial blood module for an arterial blood gas syringe in accordance with another aspect of the present invention.

FIG. 38 is partial side view of the arterial blood module of FIG. 37 in a first configuration.

FIG. 39 is a partial schematic view of the arterial blood module of FIG. 37 in the first configuration.

FIG. 40 is a partial schematic view of the arterial blood module of FIG. 37 in a second configuration.

FIG. 41 is a perspective view of an arterial blood module for an arterial blood gas syringe in accordance with another aspect of the present invention in a first configuration.

FIG. 42 is a perspective view of the arterial blood module of FIG. 41 in a second configuration.

FIG. 43 is a partial cross-sectional view of the arterial blood module of FIG. 41 in the first configuration.

FIG. 44 is a cross-sectional view of the arterial blood module of FIG. 41 in the second configuration.

FIG. 45 is a perspective view of an arterial blood module for an arterial blood gas syringe in accordance with another aspect of the present invention in a first configuration.

FIG. 46 is a perspective view of the arterial blood module of FIG. 45 in a second configuration.

FIG. 47 is a cross-sectional view of the arterial blood module of FIG. 45 in the first configuration.

FIG. 48 is a partial cross-sectional view of the arterial blood module of FIG. 45 in the second configuration.

FIG. 49 is a partial cross-sectional view of the arterial blood module of FIG. 45 in the first configuration.

FIG. 50 is an end view of an eccentric needle carrier for use with the arterial blood module of FIG. 45 in a second configuration.

FIG. 51 is an end view of the eccentric needle carrier for use with the arterial blood module of FIG. 45 in a first configuration.

FIG. 52 is a partial cross-sectional view of an arterial blood module for an arterial blood gas syringe in accordance with another aspect of the present invention in a first configuration.

FIG. 53 is a partial cross-sectional view of the arterial blood module of FIG. 52 in a second configuration.

FIG. 54 is a partial cross-sectional view of an arterial blood module for an arterial blood gas syringe in accordance with another aspect of the present invention in a first configuration.

FIG. 55 is a partial cross-sectional view of the arterial blood module of FIG. 54 in a second configuration.

FIG. 56 is an exploded view of the arterial blood module of FIG. 54.

FIG. 57 is another exploded view of the arterial blood module of FIG. 54.

FIG. 58 is a partial cross-sectional view of an arterial blood module for an arterial blood gas syringe in accordance with another aspect of the present invention in a first configuration.

FIG. 59 is a partial cross-sectional view of the arterial blood module of FIG. 58 in a second configuration.

FIG. 60 is a partial cross-sectional view of a safety shield assembly for use with an arterial blood module for an arterial blood gas syringe in accordance with another aspect of the present invention in a first configuration.

FIG. 61 is a partial cross-sectional view of a safety shield assembly of FIG. 60 in a second configuration.

FIG. 62 is a partial cross-sectional view of a safety shield assembly for use with an arterial blood module for an arterial blood gas syringe in accordance with another aspect of the present invention in a first configuration.

FIG. 63 is a partial cross-sectional view of a safety shield assembly of FIG. 62 in a second configuration.

FIG. 64 is a perspective view of an arterial blood module and arterial blood gas syringe in accordance with another aspect of the present invention in a first configuration.

FIG. 65 is a side view of the arterial blood module of FIG. 64.

FIG. 66 is a perspective view of the arterial blood module of FIG. 64.

FIG. 67 is a perspective view of the arterial blood module and arterial blood gas syringe of FIG. 64 in a second configuration.

FIG. 68 is a partial cross-sectional view of the arterial blood module of FIG. 64 in a first configuration.

FIG. 69 is a partial cross-sectional view of the arterial blood module of FIG. 64 in a second configuration.

FIG. 70 is a perspective view of an arterial blood module and arterial blood gas syringe in accordance with another aspect of the present invention in a first configuration.

FIG. 71 is a side view of the arterial blood module of FIG. 70.

FIG. 72 is a perspective view of the arterial blood module of FIG. 70.

FIG. 73 is a perspective view of the arterial blood module and arterial blood gas syringe of FIG. 70 in a second configuration.

FIG. 74 is a partial cross-sectional view of the arterial blood module of FIG. 70 in a first configuration.

FIG. 75 is a partial cross-sectional view of the arterial blood module of FIG. 70 in a second configuration.

FIG. 76 is a perspective view of an actuator for use with the arterial blood module of FIG. 70.

FIG. 77 is another perspective view of the actuator for use with the arterial blood module of FIG. 70.

FIG. 78 is a perspective view of an arterial blood module and arterial blood gas syringe in accordance with another aspect of the present invention in a first configuration.

FIG. 79 is a side view of the arterial blood module of FIG. 78.

FIG. 80 is a perspective view of the arterial blood module of FIG. 78.

FIG. 81 is a perspective view of the arterial blood module and arterial blood gas syringe of FIG. 78 in a second configuration.

FIG. 82 is a partial cross-sectional view of the arterial blood module of FIG. 78 in a first configuration.

FIG. 83 is a partial cross-sectional view of the arterial blood module of FIG. 78 in a second configuration.

FIG. 84 is a perspective view of a lever actuator and stopcock valve for use with the arterial blood module of FIG. 78.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

The present disclosure provides an arterial blood module removably connectable to an arterial blood collection element. The arterial blood module includes a housing, a needle, a cap removably securable over the needle, a mixing chamber, and a safety shield engaged with a portion of the housing and transitionable from a first shield position in which a portion of the needle is exposed to a second shield position in which the needle is shielded by a portion of the safety shield. In one embodiment, the present disclosure includes an arterial blood collection system that includes an arterial blood collection element defining a collection chamber and an arterial blood module removably connectable to a portion of the arterial blood collection element.

In an exemplary embodiment, the arterial blood collection system of the present disclosure provides a novel blood collection device for collecting of arterial blood samples using a radial stick technique. The system of the present disclosure provides an efficient system that streamlines and reduces the number of workflow steps and enables singlehanded device operation which allows for more efficient Arterial Blood Gas (ABG) collection procedures. The arterial blood module of the present disclosure includes ergonomic touch points, push button safety shield, automatic anticoagulant mixing and integrated vent cap for expelling air bubbles after a collection procedure, if necessary.

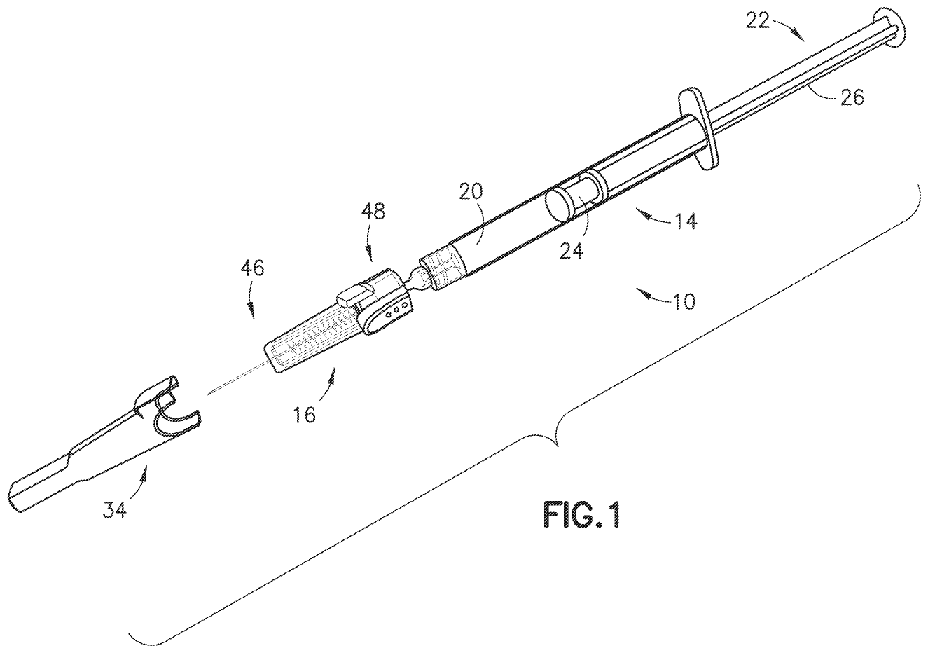

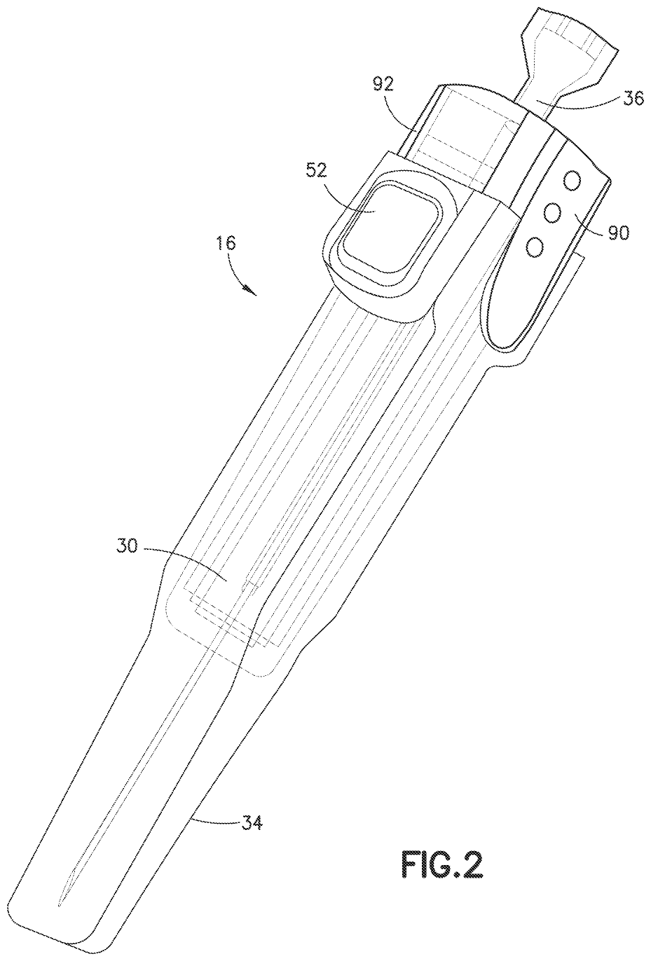

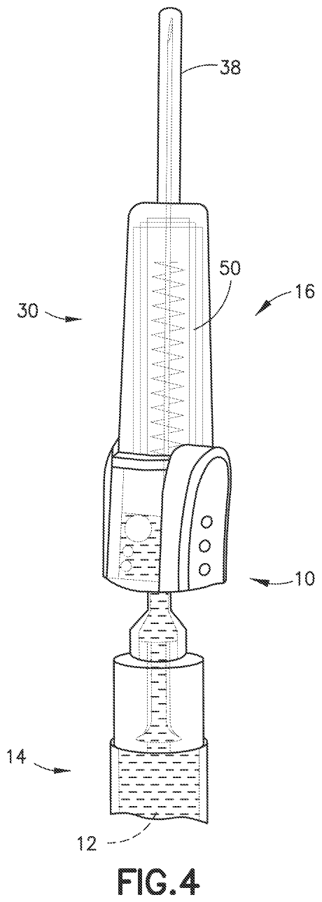

FIGS. 1-20 illustrate exemplary embodiments of an arterial blood collection system 10 of the present disclosure that is adapted to receive a biological fluid sample, such as an arterial blood sample 12. In one embodiment, the arterial blood collection system 10 of the present disclosure includes an arterial blood collection element 14 and an arterial blood module 16 that is removably connectable to a portion of the arterial blood collection element 14.

Referring to FIGS. 1 and 4-12, in one embodiment, the arterial blood collection element 14 of the present disclosure is adapted to receive a biological fluid sample, such as an arterial blood sample 12, and defines a collection chamber 20. In an exemplary embodiment, the arterial blood collection element 14 includes a plunger rod assembly 22 having a stopper 24 and a plunger rod 26. In one embodiment, the arterial blood collection element 14 is a conventional arterial blood gas syringe assembly, e.g., either a luer-lock or luer-slip arterial blood gas syringe assembly. The arterial blood module 16 of the present disclosure may be compatible with any conventional arterial blood collection elements or arterial blood gas syringe assemblies.

When using a system 10 of the present disclosure to remove arterial blood, the blood at arterial pressure is greater than the normal atmospheric or ambient pressure within the collection chamber 20 of the arterial blood collection element 14, and thus, upon inserting a needle 32 of the arterial blood module 16 into an artery, an arterial blood sample 12 will flow from the patient through the arterial blood module 16 to the collection chamber 20 of the arterial blood collection element 14, as described in more detail below. In this manner, the system 10 of the present disclosure self-fills due to the arterial blood pressure and a vented arterial blood gas syringe stopper.

Referring to FIGS. 1-20, in one embodiment, the arterial blood module 16 of the present disclosure is removably connectable to a portion of the arterial blood collection element 14 and includes a housing 30, a needle 32, a cap 34, a mixing chamber 36, a flow channel 37, a safety shield 38, and a sample stabilizer 39. In one embodiment, the housing 30 includes a first end 46 and a second end 48. In one embodiment, the housing 30 of the arterial blood module 16 defines a flow channel 37 that extends from the first end 46 to the second end 48.

In an exemplary embodiment, the arterial blood module 16 of the present disclosure is removably connectable to a portion of the arterial blood collection element 14 via conventional methods and structure. For example, in one embodiment, the arterial blood module 16 of the present disclosure is removably connectable to a portion of the arterial blood collection element 14 via a standard luer connection.

Advantageously, the arterial blood module 16 of the present disclosure allows for automatic mixing with a sample stabilizer 39. For example, in one embodiment, the mixing chamber 36 is provided in fluid communication with the flow channel 37. The mixing chamber 36 and the sample stabilizer 39 are positioned such that a biological fluid sample, such as an arterial blood sample 12, will first pass through a sample stabilizer 39, then the blood sample 12 and the sample stabilizer 39 pass through the mixing chamber 36, and subsequently the sample 12 with the sample stabilizer 39 properly mixed therein flow into the collection chamber 20 of the arterial blood collection element 14. In this way, the blood sample 12 may be mixed with a sample stabilizer 39, such as an anticoagulant or other additive, provided within the arterial blood module 16, before passing through the mixing chamber 36 for proper mixing of the sample stabilizer 39 within the blood sample 12, and then the stabilized sample is received and stored within the collection chamber 20 of the arterial blood collection element 14.

In one embodiment, a sample stabilizer 39 is disposed within the flow channel 37 between the first end 46 of the housing 30 and the mixing chamber 36. The arterial blood module 16 of the present disclosure provides passive and fast mixing of a blood sample 12 with the sample stabilizer 39. For example, the arterial blood module 16 includes a mixing chamber 36 that allows for passive mixing of the blood sample 12 with an anticoagulant or another additive, such as a blood stabilizer, as the blood sample 12 flows through the mixing chamber 36.

The sample stabilizer can be an anticoagulant, or a substance designed to preserve a specific element within the blood such as, for example, RNA, protein analyte, or other element. In one embodiment, the sample stabilizer 39 is disposed within the flow channel 37 between the first end 46 of the housing 30 and the mixing chamber 36. In other embodiments, the sample stabilizer 39 may be disposed in other areas within the housing 30 of the arterial blood module 16.

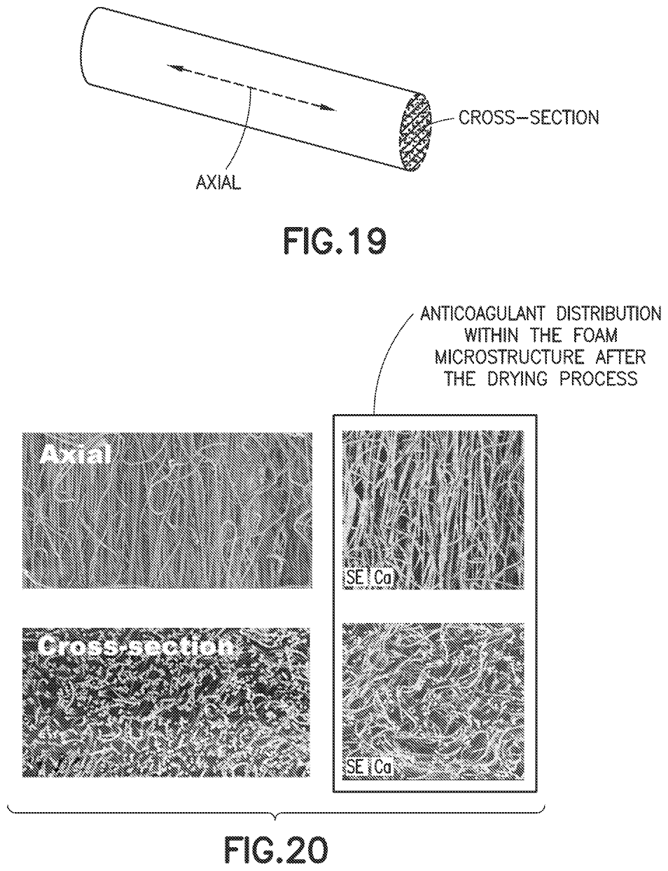

Referring to FIGS. 19-20, in one embodiment, the arterial blood module 16 includes a material 40 including pores 42 that is disposed within the flow channel 37 between the first end 46 of the housing 30 and the mixing chamber 36 and a dry anticoagulant powder 44 that is within the pores 42 of the material 40. In this manner, the arterial blood module 16 may include a dry anticoagulant, such as Heparin or EDTA, deposited on or within a portion of the arterial blood module 16. In one embodiment, the material 40 is an open cell foam that contains dry anticoagulant dispersed within the cells of the open cell foam to promote the effectiveness of the flow-through mixing and anticoagulant uptake. In one embodiment, the sample stabilizer 39 is the dry anticoagulant powder 44.

In one embodiment, the open cell foam may be treated with an anticoagulant to form a dry anticoagulant powder finely distributed throughout the pores of the open cell foam. As the blood sample 12 enters the arterial blood module 16, the blood sample 12 passes through the open cell foam and is exposed to the anticoagulant powder available throughout the internal pore structure of the open cell foam. In this manner, the sample 12 dissolves and mixes with the dry anticoagulant powder 44 while passing through the material 40 or open cell foam.

The open cell foam may be a soft deformable open cell foam that is inert to blood, for example, a melamine foam, such as Basotect® foam commercially available from BASF, or may consist of a formaldehyde-melamine-sodium bisulfite copolymer. The open cell foam may also be a flexible, hydrophilic open cell foam that is substantially resistant to heat and organic solvents. In one embodiment, the foam may include a sponge material.

The anticoagulant or other additive may be introduced into the open cell foam by soaking the foam in a liquid solution of the additive and water and subsequently evaporating the water forming a dry additive powder finely distributed throughout the internal structure of the foam.

The arterial blood module 16 includes a mixing chamber 36 that allows for passive mixing of the blood sample 12 with an anticoagulant or another additive, such as a blood stabilizer, as the blood sample 12 flows through the mixing chamber 36. In one embodiment, the mixing chamber 36 is disposed between the first end 46 and the second end 48 of the housing 30 of the arterial blood module 16. In one embodiment, the mixing chamber 36 is adjacent the second end 48.

The internal portion of the mixing chamber 36 may have any suitable structure or form as long as it provides for the mixing of the blood sample 12 with an anticoagulant or another additive as the blood sample 12 passes through the flow channel 37 of the arterial blood module 16.

The mixing chamber 36 receives the sample 12 and the sample stabilizer 39 therein and effectuates distributed mixing of the sample stabilizer 39 within the sample 12. The mixing chamber 36 effectuates distributed mixing of the sample stabilizer 39 within the sample 12 and prevents a very high sample stabilizer concentration in any portion of the blood sample 12. This prevents underdosing of the sample stabilizer 39 in any portion of the blood sample 12. The mixing chamber 36 effectuates distributed mixing of the sample stabilizer 39 within the sample 12 so that an approximately equal amount and/or concentration of the sample stabilizer 39 is dissolved throughout the blood sample 12, e.g., an approximately equal amount and/or concentration of the sample stabilizer 39 is dissolved into the blood sample 12 from a front portion of the blood sample 12 to a rear portion of the blood sample 12.

Referring to FIGS. 1-20, in one embodiment, the arterial blood module 16 includes a needle 32 extending from the first end 46 of the housing 30. In an exemplary embodiment, the needle 32 includes thin wall needle technology. For example, the thin wall needle technology of the present disclosure provides a small gage needle while still maintaining a high flow rate resulting in a fast fill time and shorter patient exposure to an uncomfortable procedure.

Referring to FIGS. 1-20, in one embodiment, the arterial blood module 16 includes a safety cap 34 that is removably securable over the needle 32. The safety cap 34 ensures the needle 32 is completely covered and shielded before any collection procedure begins. Before use of the arterial blood module 16, a user can remove the safety cap 34 from the arterial blood module 16.

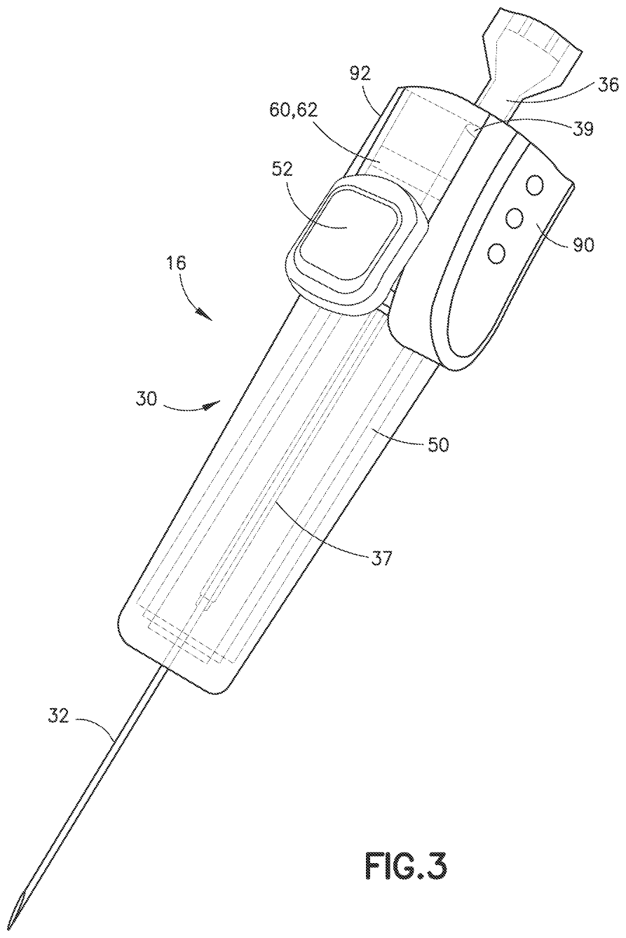

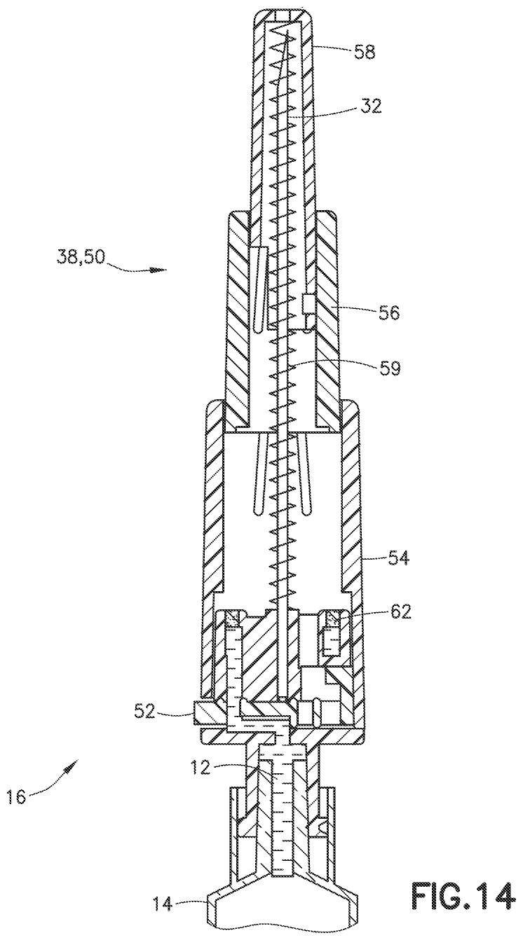

Referring to FIGS. 1-20, in one embodiment, the arterial blood module 16 includes a safety shield 38 that is engaged with a portion of the housing 30 and is transitionable from a first shield position (FIG. 3) in which a portion of the needle 32 is exposed to a second shield position (FIGS. 14, 16, and 18) in which the needle 32 is shielded by a portion of the safety shield 38.

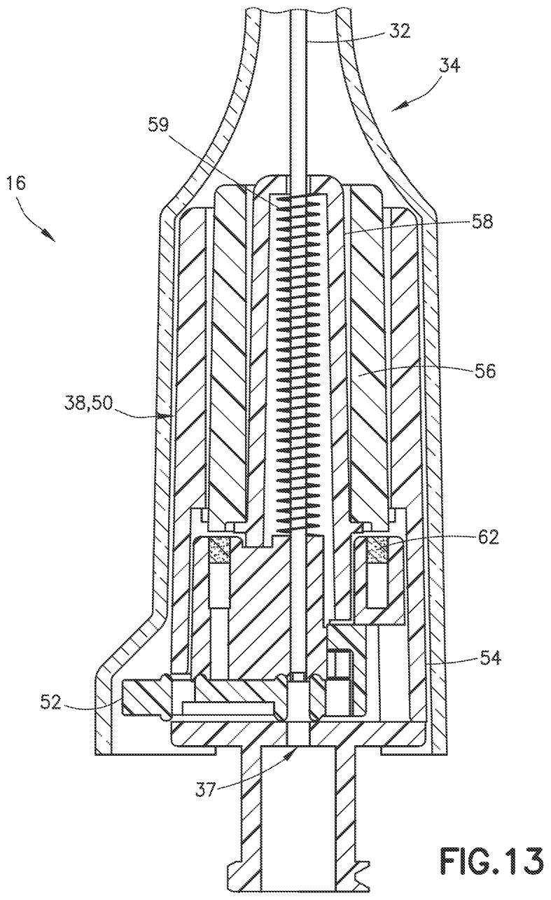

Advantageously, the safety shield 38 of the arterial blood module 16 of the present disclosure allows for automatic shielding of the needle 32 after use. For example, in one exemplary embodiment, the safety shield 38 includes a shield assembly 50 and an actuator 52 that is transitionable from a first actuator position (FIGS. 13, 15, and 17) in which the actuator 52 engages a portion of the shield assembly 50 to lock the shield assembly 50 in the first shield position (FIG. 3) to a second actuator position (FIGS. 14, 16, and 18) in which the actuator 52 releases the shield assembly 50 and the safety shield 38 automatically moves to the second shield position (FIGS. 14, 16, and 18). In one exemplary embodiment, the actuator 52 comprises a push button. In this manner, an integrated push-button safety shield 38 of the present disclosure provides a device that can be singlehanded activated by the simple push of a button after the collection procedure is complete while not obscuring a healthcare worker's view during the procedure. In this way, the safety shield 38 of the arterial blood module 16 of the present disclosure allows for automatic shielding of the needle 32 after use. For example, in one exemplary embodiment, upon activating the actuator 52 of the safety shield 38, a spring 59 exerts a force on a portion of the shield assembly 50 to automatically move the safety shield 38 to the second shield position in which the needle 32 is shielded by a portion of the safety shield 38.

Referring to FIGS. 13-18, in exemplary embodiments, the shield assembly 50 includes telescoping shields. For example, referring to FIGS. 13-14, in one exemplary embodiment, the shield assembly 50 includes a fixed outer shield 54; a middle movable shield 56 in communication with the fixed outer shield 54, wherein the middle movable shield 56 moves relative to the fixed outer shield 54; and an inner movable shield 58 in communication with the middle movable shield 56, wherein the inner movable shield 58 moves relative to the middle movable shield 56.

Referring to FIG. 13, in one embodiment, with the safety shield 38 in the first shield position, the inner movable shield 58 is nested inside the middle movable shield 56, and the middle movable shield 56 is nested inside the fixed outer shield 54.

Referring to FIG. 14, in one embodiment, with the safety shield 38 in the second shield position, the inner movable shield 58 extends from the middle movable shield 56, and the middle movable shield 56 extends from the fixed outer shield 54.

In one exemplary embodiment, upon activating the actuator 52 of the safety shield 38, a spring 59 exerts a force on a portion of the shield assembly 50 to automatically move the safety shield 38 to the second shield position in which the needle 32 is shielded by a portion of the safety shield 38. For example, referring to FIG. 13, in a first position, the spring 59 is disposed within the inner shield 58 in a compressed position. Referring to FIG. 14, upon activating the actuator 52 of the safety shield 38, the spring 59 is able to exert the stored force on a portion of the inner shield 58 to automatically move the telescoping shields 54, 56, 58 to the second shield position in which the needle 32 is shielded by a portion of the shield assembly 50.

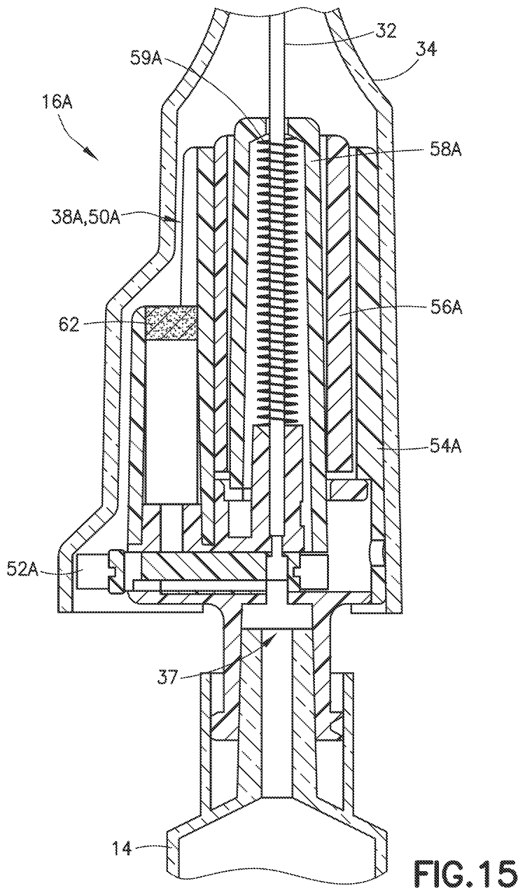

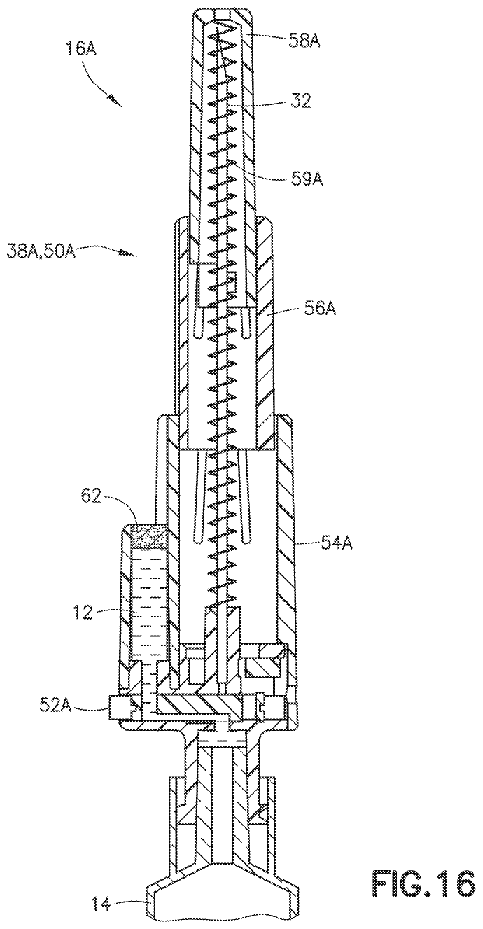

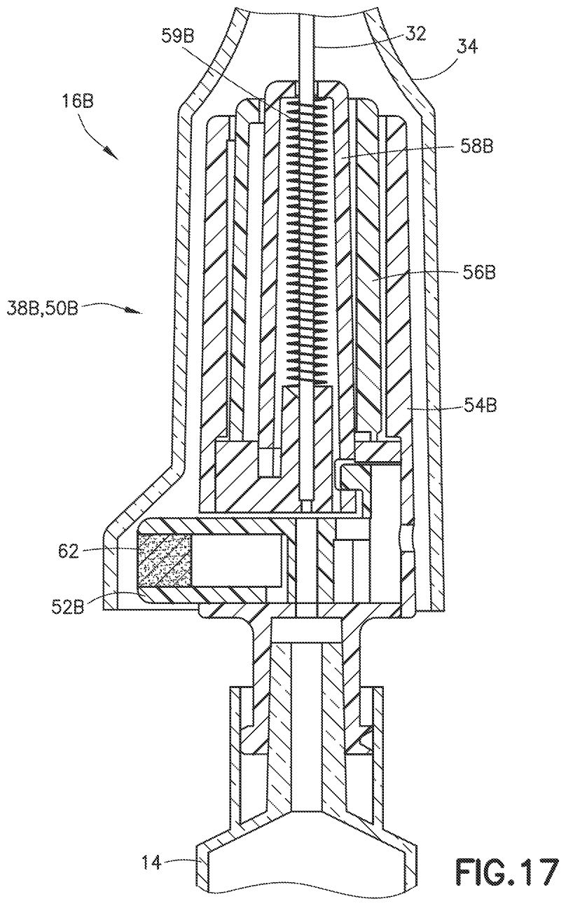

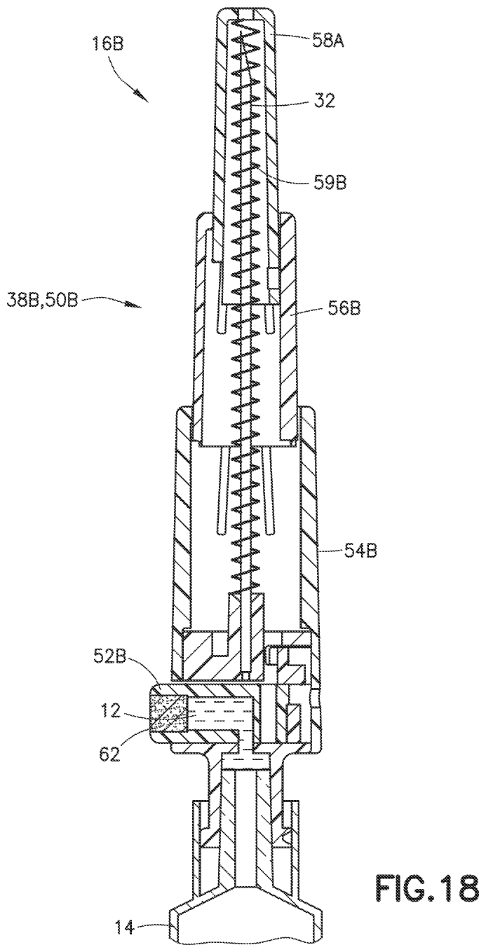

FIGS. 15-18 illustrate other exemplary embodiments. The embodiment illustrated in FIGS. 15-16 includes similar components to the embodiment illustrated in FIGS. 13-14, and the similar components are denoted by a reference number followed by the letter A. The embodiment illustrated in FIGS. 17-18 also includes similar components to the embodiment illustrated in FIGS. 13-14, and the similar components are denoted by a reference number followed by the letter B. For the sake of brevity, these similar components and the similar steps of using arterial blood module 16A (FIGS. 15-16) and arterial blood module 16B (FIGS. 17-18) will not all be discussed in conjunction with the embodiments illustrated in FIGS. 15-16 and FIGS. 17-18. Advantageously, the arterial blood module 16 of the present disclosure allows for an integrated air venting system which allows for the removal of trapped air bubbles by simply expelling it into a vented compartment inside the arterial blood module 16. For example, in one exemplary embodiment, the arterial blood module 16 includes a vent chamber 60, a venting plug 62, a first valve 64, and a second valve 66.

In one embodiment, the housing 30 of the arterial blood module 16 defines a vent chamber 60. In an exemplary embodiment, the arterial blood module 16 includes a venting plug 62 that allows air to pass therethrough and prevents a blood sample 12 from passing therethrough, wherein a portion of the venting plug 62 is in communication with the vent chamber 60. In this manner, any air bubbles contained within the arterial blood collection element 14 can be expelled outside of the system 10 through the vent chamber 60 and out the venting plug 62. The construction of the arterial blood module 16, the vent chamber 60, and the venting plug 62 allows air to pass through the arterial blood module 16 while preventing the blood sample 12 from passing through the arterial blood module 16 and may include a hydrophobic filter.

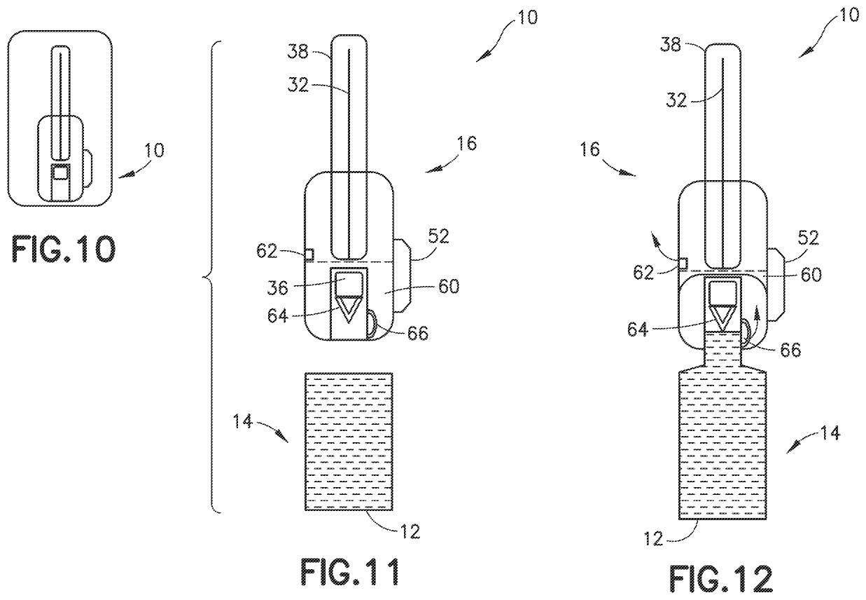

Referring to FIGS. 10-12, in one embodiment, the arterial blood module 16 includes a first valve 64. In an exemplary embodiment, with the arterial blood module 16 connected to the arterial blood collection element 14, the blood sample 12 enters the collection chamber 20 of the arterial blood collection element 14 via the needle 32 and the first valve 64. The first valve 64 allows the blood sample 12 to pass from the arterial blood module 16 to the collection chamber 20 of the arterial blood collection element 14. The first valve 64 blocks the blood sample 12 from passing from the collection chamber 20 of the arterial blood collection element 14 back to the arterial blood module 16. In one embodiment, the first valve 64 is a one-way valve.

In one embodiment, the arterial blood module 16 includes a second valve 66. In an exemplary embodiment, with the arterial blood module 16 connected to the arterial blood collection element 14, air contained in the collection chamber 20 of the arterial blood collection element 14 and a portion of the blood sample 12 enter the vent chamber 60 via the second valve 66. In one embodiment, the second valve 66 is a one-way valve.

Advantageously, the arterial blood module 16 of the present disclosure provides an ergonomic design with designated touch points to facilitate precise and easy handling of the arterial blood module 16 during a collection procedure. For example, in one exemplary embodiment, referring to FIGS. 1-20, the arterial blood module 16 includes a first finger grip portion 90. In another exemplary embodiment, the arterial blood module 16 includes a second finger grip portion 92, wherein the first finger grip portion 90 is disposed opposite the second finger grip portion 92.

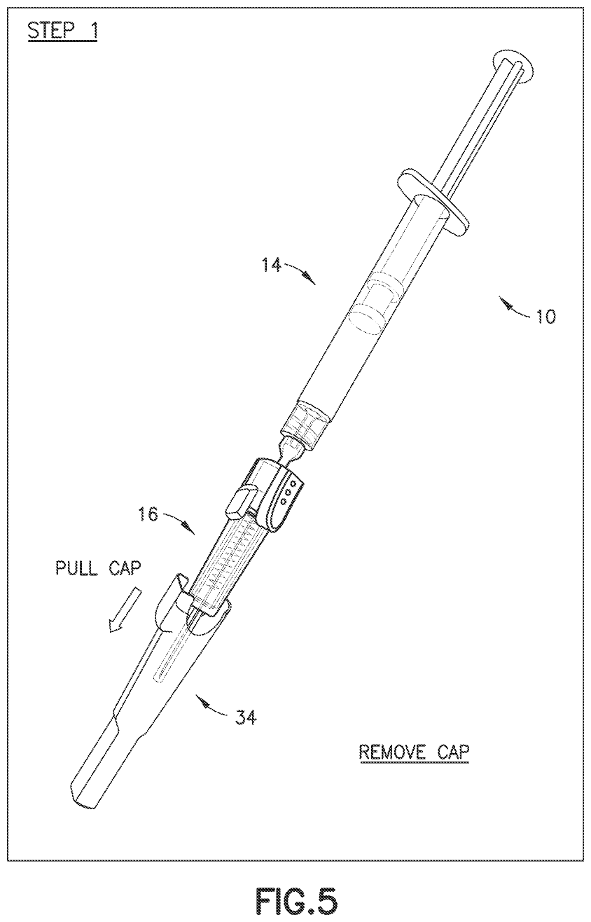

Referring to FIGS. 5-9, use of an arterial blood collection system 10 of the present disclosure having an arterial blood module 16 and an arterial blood collection element 14 will now be described.

Referring to FIG. 5, a safety cap 34 ensures that the needle 32 of the arterial blood module 16 is completely covered and shielded before any collection procedure begins. Before use of the arterial blood module 16, a user can remove the safety cap 34 by pulling it away from the arterial blood module 16 as shown in FIG. 5.

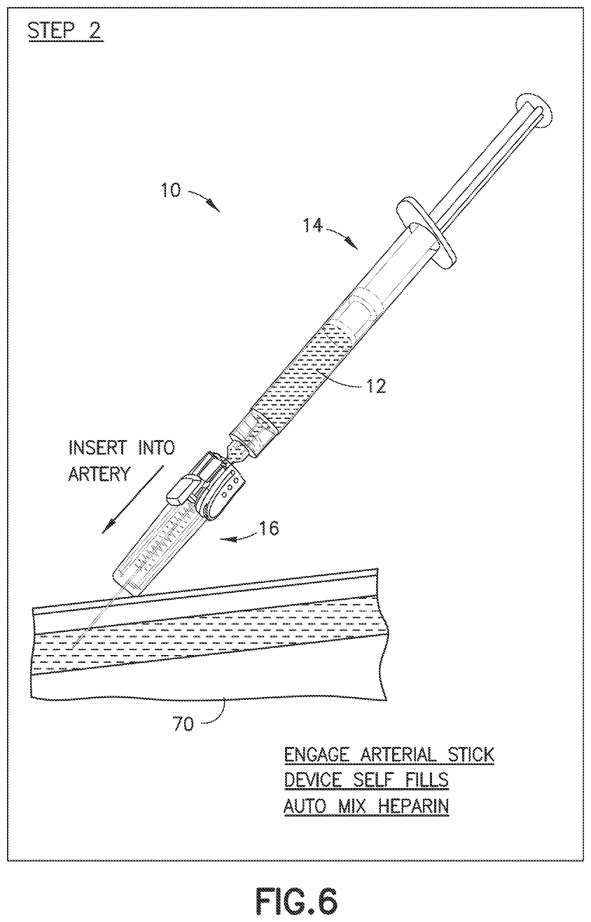

Referring to FIG. 6, the needle 32 of the system 10 is inserted into an artery 70 of a patient. The arterial blood collection element 14 of the arterial blood collection system 10 self-fills due to the arterial blood pressure and vented arterial blood gas syringe stopper as described above. An arterial blood sample 12 is automatically mixed with a sample stabilizer 39, as described above, as the blood sample 12 automatically travels from the artery 70 of the patient to the collection chamber 20 of the arterial blood collection element 14 via the arterial blood module 16.

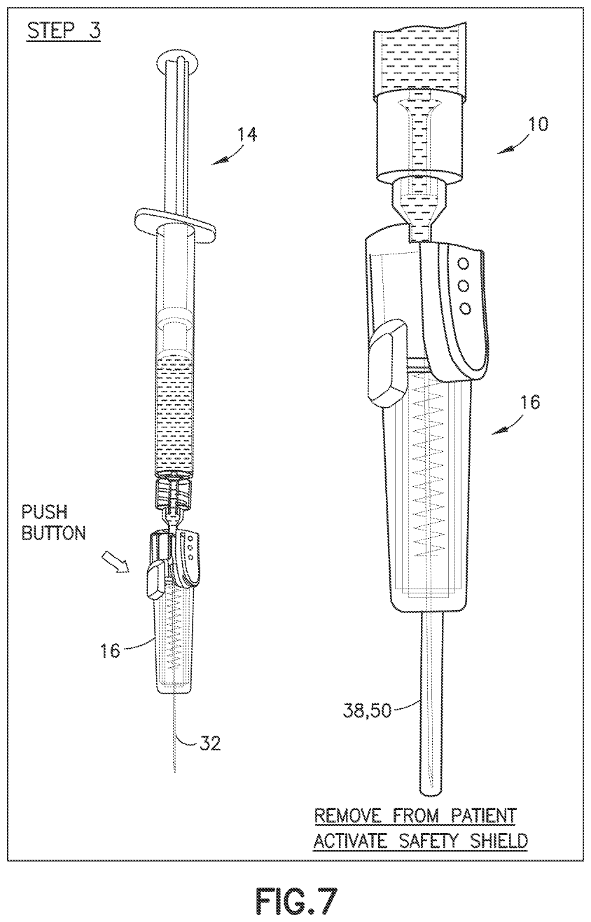

Referring to FIG. 7, the needle 32 is then removed from the artery 70 of the patient and the safely shield 38 is activated by pressing the actuator 52 which automatically shields the needle 32 with the safety shield 38. In this manner, the safety shield 38 is locked into a shielded position safely covering the needle 32 for safe handling and disposal of the arterial blood module 16.

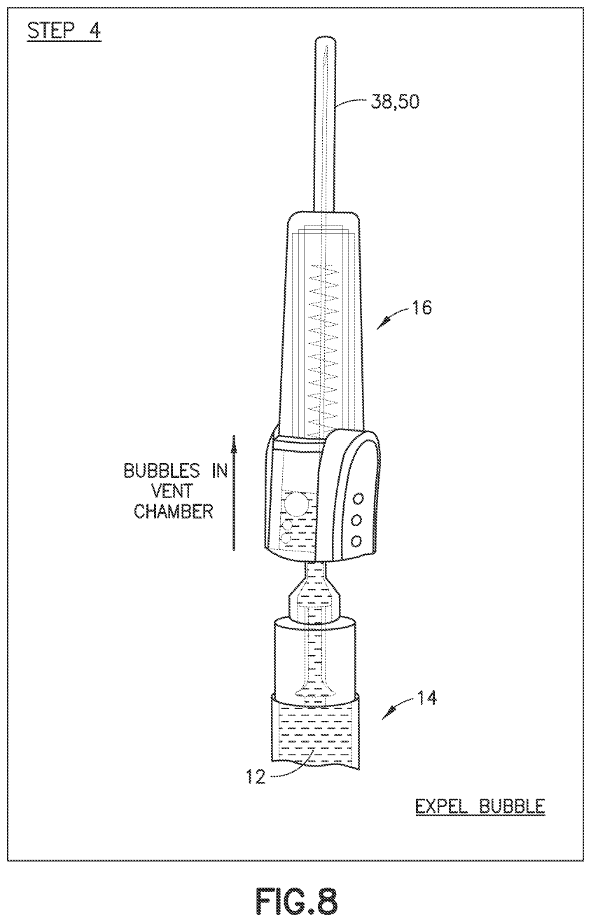

Referring to FIG. 8, the arterial blood collection system 10 is then orientated vertically by pointing the safely shielded needle 32 in an upward configuration. Next, a user can push the plunger rod 26 of the arterial blood collection element 14 to expel any trapped air bubbles from the collection chamber 20 of the arterial blood collection element 14 and out the arterial blood module 16 via the vent chamber 60 and the venting plug 62, as described in detail above.

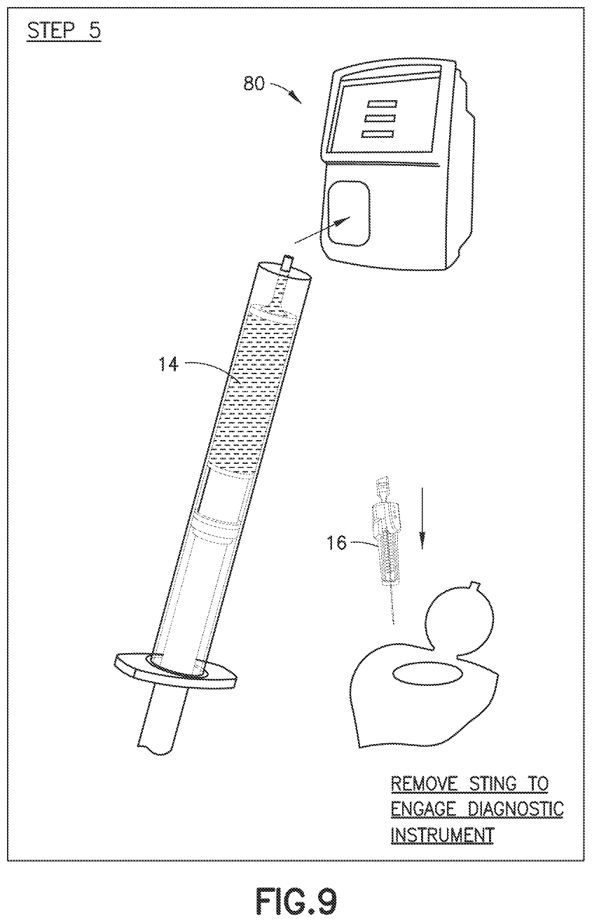

Referring to FIG. 9, the arterial blood module 16 is then removed from the arterial blood collection element 14. In this manner, the arterial blood module 16 can be safely disposed of and the arterial blood collection element 14 can be interfaced with an arterial blood gas analysis instrument 80 for sample transfer and analysis.

The present disclosure provides an arterial blood module removably connectable to an arterial blood collection element. The arterial blood module includes a housing, a needle, a cap removably securable over the needle, a mixing chamber, and a safety shield engaged with a portion of the housing and transitionable from a first shield position in which a portion of the needle is exposed to a second shield position in which the needle is shielded by a portion of the safety shield. In one embodiment, the present disclosure includes an arterial blood collection system that includes an arterial blood collection element defining a collection chamber and an arterial blood module removably connectable to a portion of the arterial blood collection element.

In an exemplary embodiment, the arterial blood collection system of the present disclosure provides a novel blood collection device for collecting of arterial blood samples using a radial stick technique. The system of the present disclosure provides an efficient system that streamlines and reduces the number of workflow steps and enables singlehanded device operation which allows for more efficient Arterial Blood Gas (ABG) collection procedures. The arterial blood module of the present disclosure includes ergonomic touch points, push button safety shield, automatic anticoagulant mixing and integrated vent cap for expelling air bubbles after a collection procedure, if necessary.

In an exemplary embodiment, the arterial blood module 16 of the present disclosure includes the open cell foam material 40. In other exemplary embodiments, the arterial blood collection element 14 could include the open cell foam material 40.

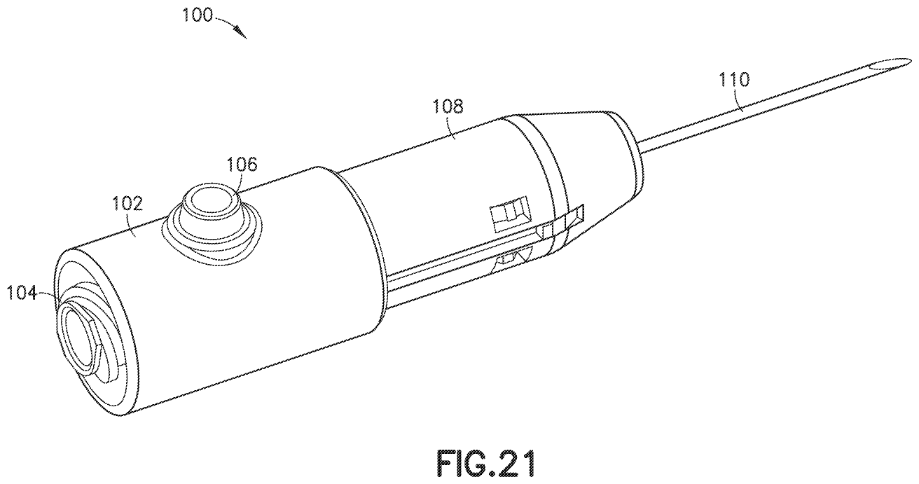

Referring now to FIGS. 21-24, an arterial blood module 100 in accordance with aspect of the present disclosure is illustrated. Arterial blood module 100 includes a hub body 102 having a proximal coupling interface 104, with the proximal coupling interface 104 being configured to removably couple the arterial blood module 100 to an arterial blood collection element such as, e.g., arterial blood collection element 14 described above. Arterial blood module 100 also includes safety shield 108, which may be selectively deployed by a user to cover a needle 110. As will be described in further detail below, deployment of the safety shield 108 is initiated by an actuator 106 housed within the hub body 102.

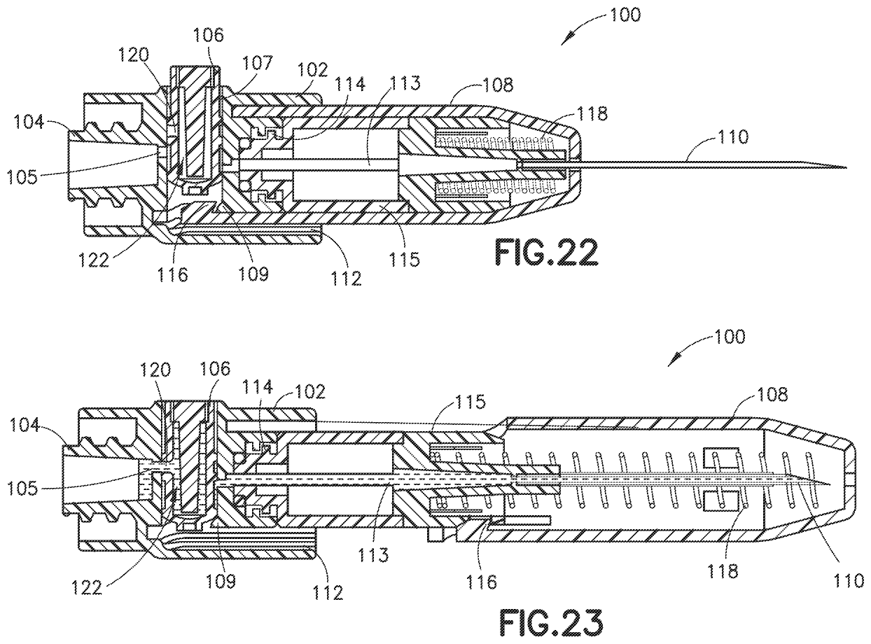

FIG. 22 illustrates the arterial blood module 100 in a first configuration, where the safety shield 108 is in an undeployed position such that the needle 110 is at least partially exposed. In this first configuration, the safety shield 108 may be releasably secured to the hub body 102 via an interface between a protruding portion 109 of the hub body 102 and a detent portion 116 of the safety shield 108. The proximal portion of safety shield 108 including the detent portion 116 is configured to be received within a slot 112 of the hub body 102. The slot 112 is in communication with an actuator bore 107 formed within the hub body 102, with the actuator 106 being slidable within the actuator bore 107 in a direction substantially transverse to the direction of slot 112 and, thus, substantially transverse to the direction of deployment of the safety shield 108. Accordingly, when the actuator 106 is depressed, a base portion of actuator 106 contacts the proximal portion of the safety shield 108, disengaging the detent portion 116 of the safety shield 108 from the protruding portion 109 of the hub body 102 such that the safety shield 108 is telescopically deployed over the needle 110 by way of spring force applied by a spring 118, as is shown in FIG. 23.

Referring again to FIG. 22, when the safety shield 108 is in an undeployed position (i.e., with the needle 110 is exposed), the arterial blood module 100 is configured to provide a fluid path between the needle 100 and an opening 105 in communication with the proximal coupling interface 104. More specifically, the arterial blood module 100 includes a needle assembly body 115 coupled to the needle 100, with the needle assembly body 115 having a flow channel 113 passing therethrough, wherein the flow channel 113 is in fluid communication with the needle 110. While not shown, the flow channel 113 may be in communication with, e.g., a sample stabilizer. In this configuration, a blood sample may flow through the needle 110, the flow channel 113, and around the actuator 106 to the opening 105, where the sample may be drawn into, e.g., an arterial blood collection element coupled to the proximal coupling interface 104.

Conversely, when the actuator 106 is depressed to deploy the safety shield 108 (as shown in FIG. 23), the fluid pathway between the opening 105 and the flow channel 113 is also disconnected, thereby stopping fluid flow between the needle 110 and the opening 105. More specifically, depression of the actuator 106 within the actuator bore 107 aligns an orifice 120 formed in the actuator 106 with the opening 105. The orifice 120 is in fluid communication with a vent chamber 122 formed within the actuator 106. The actuator 106 may also include a venting plug in communication with the vent chamber 122 that allows air to pass therethrough, while preventing a blood sample from passing therethrough. In some embodiments, the venting plug may include a hydrophobic filter. In this manner, any air bubbles contained within an arterial blood collection element coupled to the arterial blood module 100 can be expelled outside of the system through the vent chamber 122 and out the venting plug formed as part of the actuator 106.

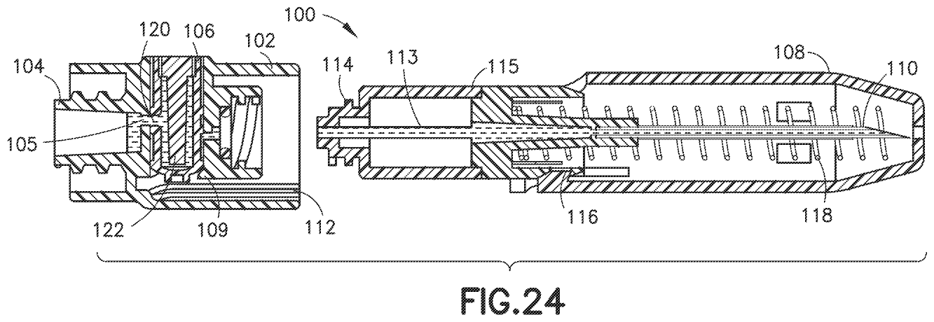

Additionally, referring to FIG. 24, arterial blood module 100 is also configured such that the needle assembly body 115, safety shield 108, and needle 110 are removably coupled to the hub body 102. For example, needle assembly body 115 may include a threaded interface 114 capable of being coupled to a corresponding threaded interface on hub body 102. In this way, the needle assembly body 115, safety shield 108, and needle 110 may together be removed and discarded into an appropriate sharps container after use, while the hub body 102 may remain coupled to an arterial blood collection element for venting of air bubbles, transport, etc.

However, while needle assembly body 115, safety shield 108, and needle 110 are removably coupled to the hub body 102, removal of these components from the hub body 102 is prevented when the safety shield 108 is in undeployed position (i.e., when the needle 110 is unprotected by the safety shield 108). As shown in FIG. 22, the interface between the detent portion 116 of the safety shield 108 from the protruding portion 109 of the hub body 102 within the slot 112 not only holds the safety shield 108 in an undeployed position, but also acts to prevent rotation of the safety shield 108 (and, thus, the needle assembly body 115) relative to the hub body 102. Accordingly, in order to remove the needle assembly body 115 and needle 110 for proper disposal, the user must first deploy the safety shield 108 by depressing the actuator 106, thereby decoupling the detent portion 116 of the safety shield 108 from the protruding portion 109 of the hub body 102 to enable rotation of the needle assembly body 115 relative to the hub body 102, as shown in FIG. 23.

Next, referring to FIGS. 25-27, an arterial blood module 150 in accordance with another aspect of the present disclosure is illustrated. Arterial blood module 150 includes a twist-off safety shield assembly 154, which is removably coupled to a hub body 152, with hub body 152 being couplable to, e.g., an arterial blood collection element 14 by way of a threaded luer coupling. The twist-off safety shield assembly 154 may also be threadably coupled to the hub body 152, but removal of the twist-off safety shield assembly 154 may require an opposite direction of rotation (e.g., a left-handed thread) than that of the interface between the hub body 152 and the arterial blood collection element 14 in order to avoid undesired detachment of the hub body 152 from the arterial blood collection element 14 when removing the twist-off safety shield assembly 154.

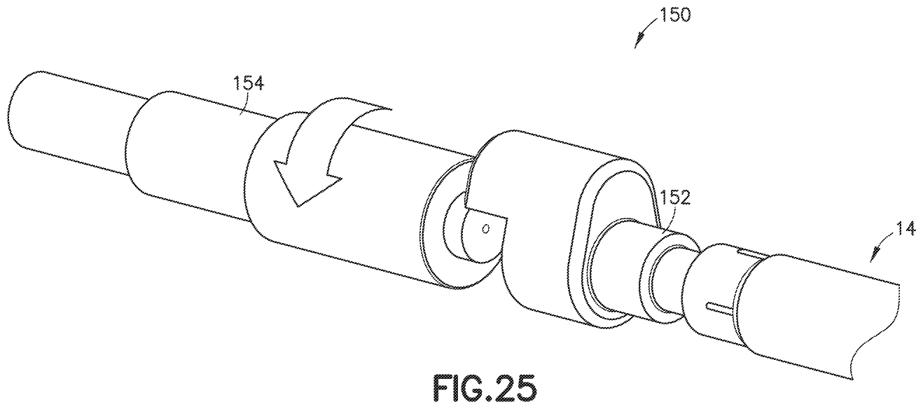

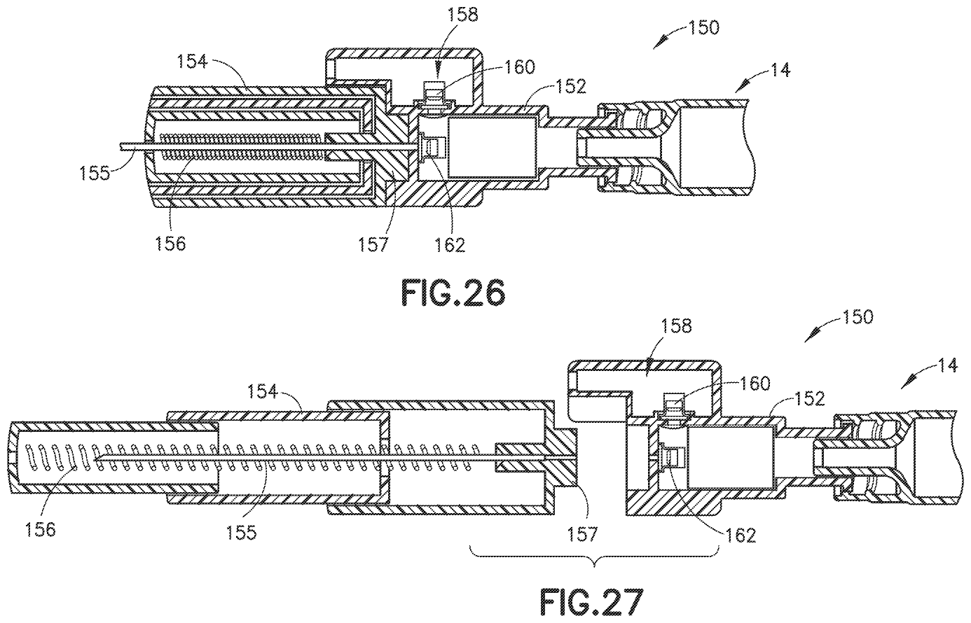

As shown in FIGS. 26 and 27, the safety shield assembly 154 includes the telescopic safety shield, needle 155, and spring 156 for biasing the respective members of the telescopic safety shield toward a deployed position, as shown in FIG. 27. The telescopic safety shield may be deployed by any appropriate method such as a push-button, twist, etc. Furthermore, an interface portion 157 is provided on a distal end of the safety-shield assembly 154, with the interface portion 157 configured to removably engage with the hub body 152 and provide a fluid communication path between the hub body 152 and the needle 155.

Hub body 152 includes a first check valve 162, a second check valve 160, and a vent chamber 158. Vent chamber 158 may include a venting plug in communication with the vent chamber 158 that allows air to pass therethrough, while preventing a blood sample from passing therethrough. In some embodiments, the venting plug may include a hydrophobic filter.

When the safety shield assembly 154 is coupled to the hub body 152, as is shown in FIG. 26, the needle 155 is fluidly coupled to the arterial blood collection element 14, with the first check valve 162 being opened in order to allow fluid to pass therethrough. However, when the safety shield assembly 154 is detached from the hub body 152, as is shown in FIG. 27, the first check valve 162 may be closed in order to prevent blood leakage and minimize air exposure. The safety shield assembly 154 may then be discarded in an appropriate sharps container.

Furthermore, in the configuration shown in FIG. 27, the plunger rod (not shown) of arterial blood collection element 14 can be depressed to expel any air bubbles contained within an arterial blood collection element 14 through the second check valve 160 and into the vent chamber 158, with the vent chamber 158 also sized and configured to hold a small volume of clotted blood sample from proximate the luer tip. As noted above, the first check valve 162 is substantially sealed when the safety shield assembly 154 is detached in order to prevent blood leakage and minimize air exposure.

By enabling safety shield assembly 154 to be easily detached from the hub body 152, the needle 155 is removed prior to transport and sample analysis, thereby minimizing the risk of needle stick injuries and/or inappropriate disposal. Furthermore, removal of the safety shield assembly 154 also reduces the overall size of the device, which may be beneficial during transport and analysis.

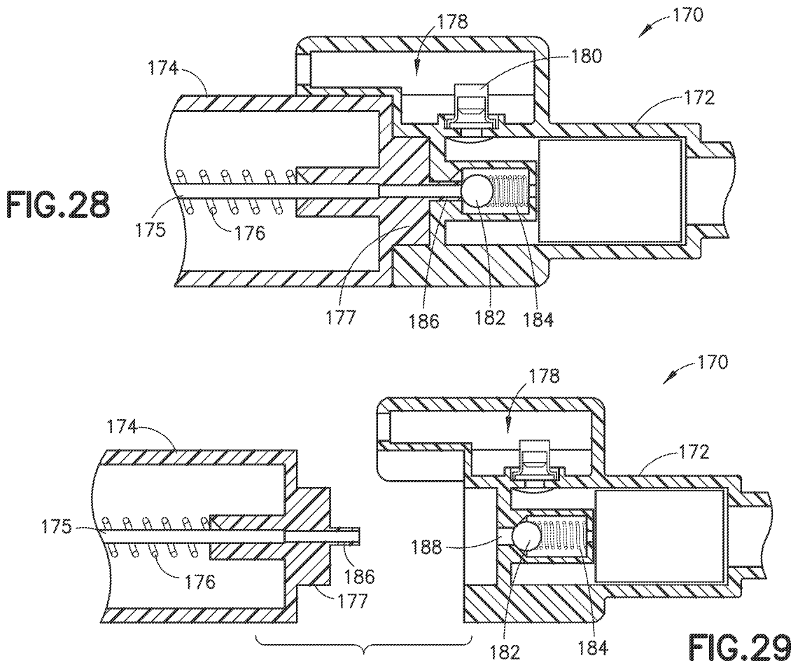

Next, referring to FIGS. 28 and 29, an arterial blood module 170 in accordance with another aspect of the present disclosure is illustrated. Similar to arterial blood module 150 described above with respect to FIGS. 25-27, arterial blood module 170 includes a twist-off safety shield assembly 174, which is removably coupled to a hub body 172, with hub body 172 being couplable to, e.g., an arterial blood collection element (not shown) by way of a threaded luer coupling. The twist-off safety shield assembly 174 may also be threadably coupled to the hub body 172, but removal of the twist-off safety shield assembly 174 may require an opposite direction of rotation (e.g., a left-handed thread) than that of the interface between the hub body 172 and the arterial blood collection element.

As is partially shown in FIGS. 28 and 29, the safety shield assembly 174 includes the telescopic safety shield, needle 175, and spring 176 for biasing the respective members of the telescopic safety shield toward a deployed position. The telescopic safety shield may be deployed by any appropriate method such as a push-button, twist, etc. Furthermore, an interface portion 177 is provided on a distal end of the safety-shield assembly 174, with the interface portion 177 configured to removably engage with the hub body 172 and provide a fluid communication path between the hub body 172 and the needle 175. Interface portion 177 further includes a distal protrusion 186, which is sized and configured to extend within (and partially beyond) an opening 188 formed in the hub body 172 when the safety shield assembly 174 is coupled to the hub body 172. The distal protrusion 186 is configured to provide a fluid pathway between the needle 175 and the hub body 172.

Hub body 172 also includes a normally-closed ball valve 182, with the ball-valve 182 biased into a normally closed position by a spring 184. As shown in FIG. 28, when the safety shield assembly 174 is coupled to the hub body 172, the distal protrusion 186 acts against the spring 184, thereby opening the ball valve 182 to allow blood to flow from the needle 175 to the hub body 172. However, when the safety shield assembly 174 is detached from the hub body 172, the distal protrusion 186 no longer acts against the spring 184, allowing the spring 184 to close the ball valve 182, thereby blocking the flow of blood from the opening 188 of the hub body 172.

Referring still to FIGS. 28 and 29, the hub body 172 further includes a check valve 180 and a vent chamber 178. Vent chamber 178 may include a venting plug in communication with the vent chamber 178 that allows air to pass therethrough, while preventing a blood sample from passing therethrough. In some embodiments, the venting plug may include a hydrophobic filter. When the safety shield assembly 174 is detached from the hub body 172, as is shown in FIG. 29, the ball valve 182 is closed in order to prevent blood leakage and minimize air exposure. Furthermore, the plunger rod (not shown) of the arterial blood collection element can be depressed to expel any air bubbles contained within an arterial blood collection element through the check valve 180 and into the vent chamber 178, with the vent chamber 178 also sized and configured to hold a small volume of clotted blood sample from proximate the luer tip.

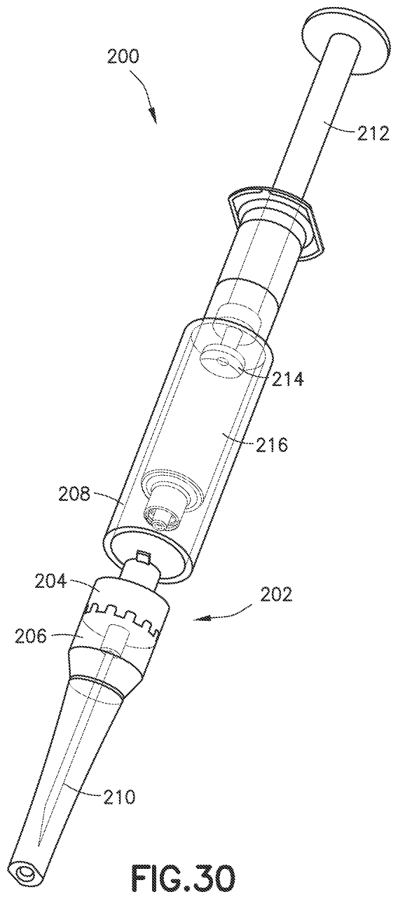

Referring now to FIGS. 30-36, an arterial blood gas syringe assembly 200 and associated componentry in accordance with another aspect of the present disclosure are illustrated. As shown in FIG. 30, arterial blood gas syringe assembly 200 includes a syringe body 216, concave plunger stopper 214, and vented plunger 212. A distal end of the syringe body 216 is configured to have, e.g., a luer connector.

Coupled to the distal end of syringe body 216 is an arterial blood module 202, with the arterial blood module 202 comprising an integrated mixing portion 206 and safety shield 208, as well as a vented cap portion 204. The vented cap portion 204 is configured to be removably couplable to the syringe body 216 by way of, e.g., a luer connection, while the integrated mixing portion 206 and safety shield 208 are configured to be removably couplable to the vented cap portion 204.

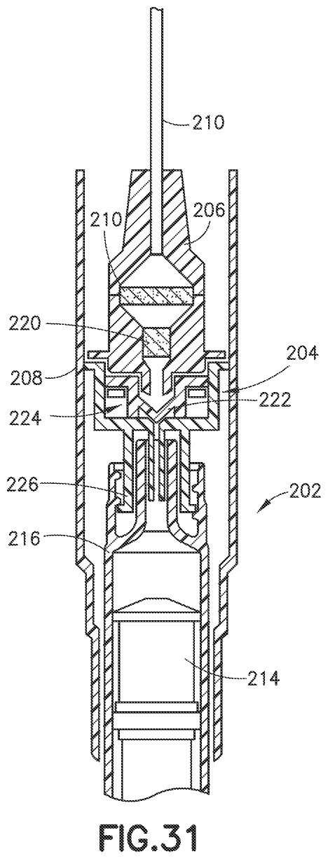

Referring to FIG. 31, the mixing portion 206 includes a needle 210 extending distally therefrom, along with filters 218, 220 positioned within a mixing chamber. While not shown, it is to be understood that a sample stabilizer may be provided within the mixing chamber of the mixing portion 206. The safety shield 208 is configured to be coupled to the mixing portion 206, and may be telescopically deployed by any appropriate method such as, e.g., a push-button, twist, etc. The vented cap portion 204 includes a one-way valve 222, a vent chamber 224, and a proximal coupling portion 226, wherein the proximal coupling portion 226 is configured to allow attachment of the vented cap portion 204 to the syringe body 216. The vent chamber 224 may include a venting plug or filter in communication with the vent chamber 224 that allows air to pass therethrough, while preventing a blood sample from passing therethrough. In some embodiments, the venting plug or filter may include a hydrophobic filter.

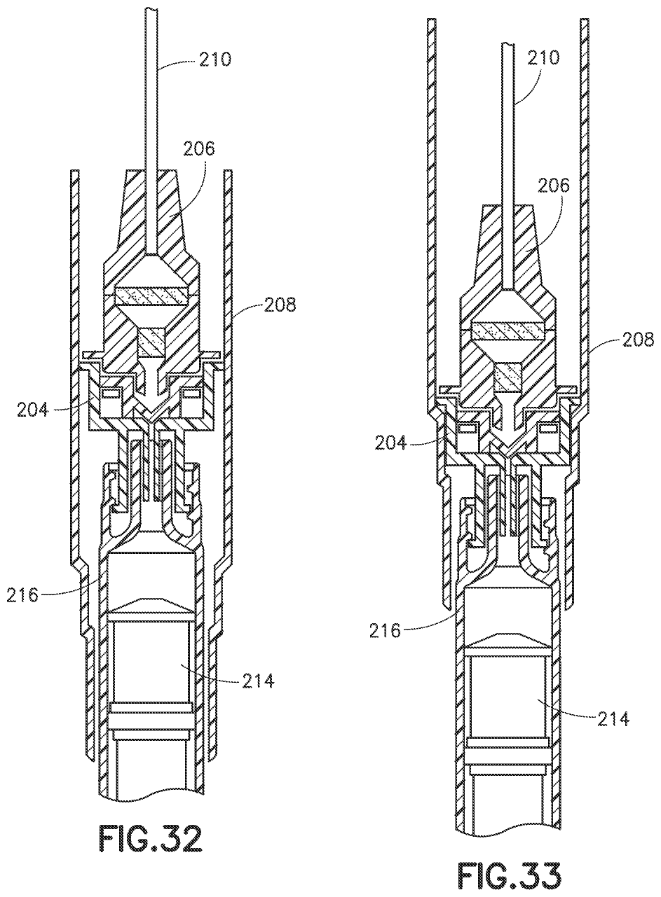

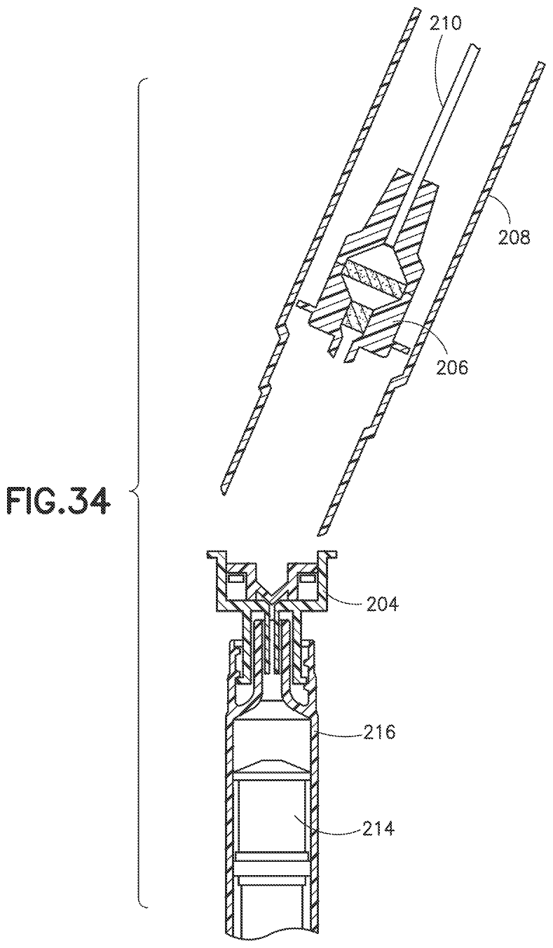

Next, referring to FIGS. 32-36, the steps of use of the arterial blood gas syringe assembly 200 in accordance with an aspect of the present disclosure are shown. First, as shown in FIG. 32, with the safety shield 208 in an undeployed position, blood is drawn through the needle 210 into the syringe body 216 via, e.g., arterial pressure. The blood sample passes through the mixing portion 206 and the one-way valve 222 of the vent cap portion 204, providing for a stabilized blood sample in the syringe body 216. Then, as shown in FIG. 33, the safety shield 208 is deployed so as to substantially cover the needle 210. With the needle 210 protected by the safety shield 208, the integrated mixing portion 206, safety shield 208, and needle 210 are together removed from the vented cap portion 204, enabling the mixing portion 206, safety shield 208, and needle 210 to be disposed of in an appropriate sharps container, as shown in FIG. 34.

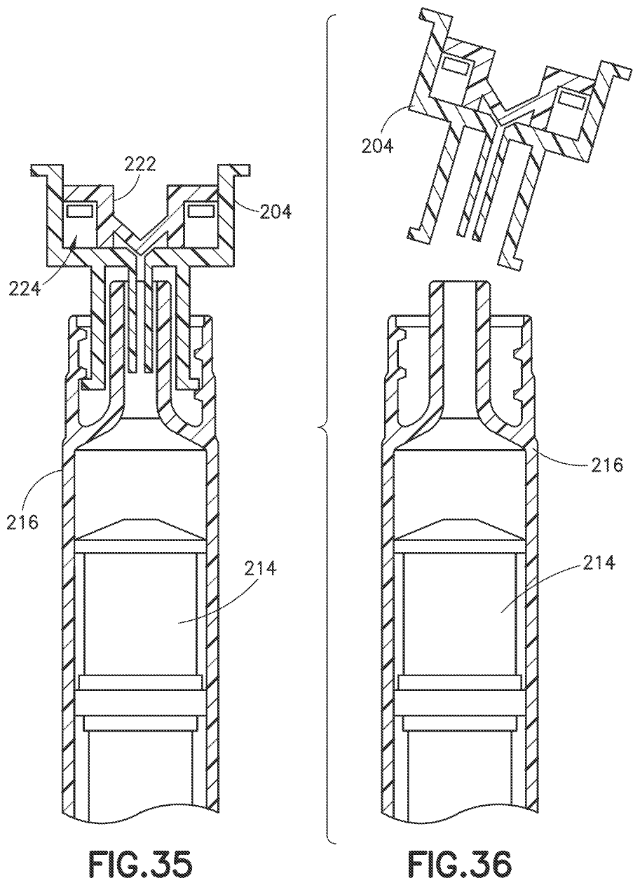

Next, referring to FIG. 35, the syringe body 216 can be held in an upright position to move any air bubbles within the sample to rise toward the distal end, and the plunger rod 212 (shown in FIG. 30) can be depresses to move the concave plunger stopper 214 distally, thereby expelling the air bubbles and a small volume of clotted blood sample into the vent chamber 224 of the vented cap portion 204. The one-way valve 222 of the vented cap portion 204 substantially prevents leakage of blood from the vented cap portion 204 during this venting step. Finally, referring to FIG. 36, the vented cap portion 204 containing the clotted blood sample and/or air bubbles can be removed from the syringe body 216 and discarded in an appropriate container, and the remaining blood sample within the syringe body 216 can be delivered for analysis.

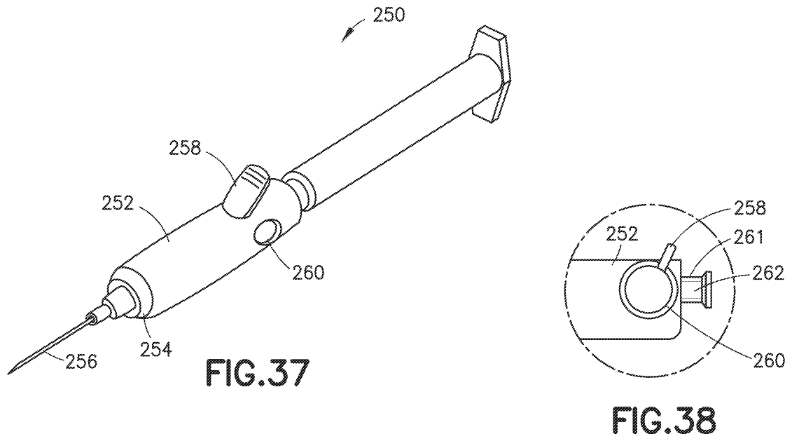

Referring now to FIGS. 37-40, an arterial blood module 250 in accordance with another aspect of the present disclosure is illustrated. As shown in FIG. 37, the arterial blood module 250 is configured to be coupled to, e.g., an arterial blood collection element 14 by way of a threaded luer coupling or similar interface. The arterial blood module 250 includes a body 252, a safety shield 254 (e.g., a telescopic shield), and a needle 256. Additionally, a rotatable stopcock 260 is provided within a proximal end portion of the body 252, with the rotatable stopcock 260 being configured for partial rotation by an actuator 258 disposed at least partially outside the body 252. As shown in FIG. 38, a proximal connector 261 is provided on the body 252, with the proximal connector 261 being configured for removable coupling to, e.g., a luer connector of the arterial blood collection element 14. A passage 262 passes through the proximal connector 261 to provide a fluid passage between the rotatable stopcock 260 and the arterial blood collection element 14.

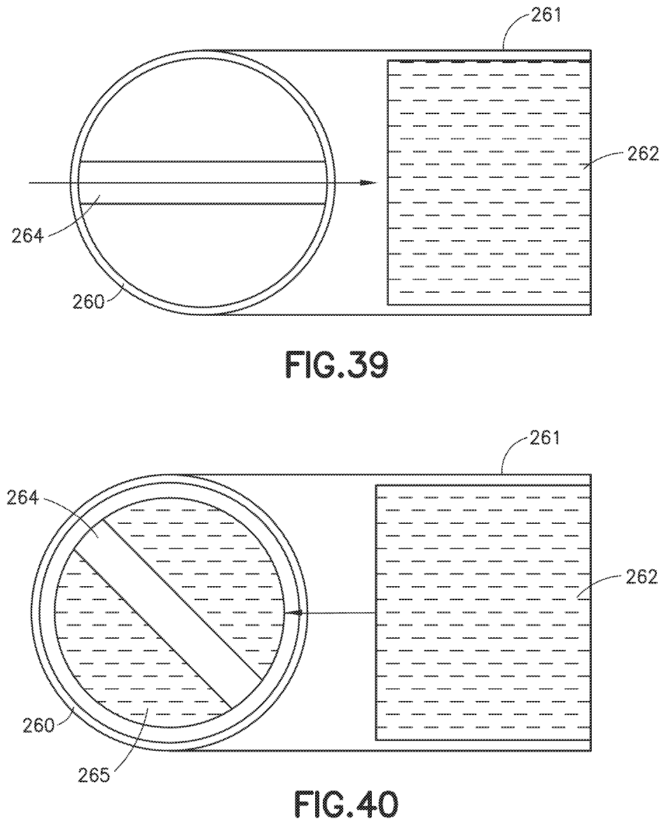

Referring to FIG. 39, when the rotatable stopcock 260 is in a first configuration (i.e., the actuator 258 is undepressed), a blood sample is configured to pass through a channel 264 in the rotatable stopcock 260, passing from the needle 256 and passage 262 to the arterial blood collection element 14 in order to collect a blood sample. However, after the desired blood sample is collected within the arterial blood collection element 14, the actuator 258 can be depressed, thereby partially rotating the rotatable stopcock 260 such that the channel 264 is angled away from the needle 256 and passage 262, as is shown in FIG. 40. Instead, a second opening (not shown) in the rotatable stopcock 260 may be aligned with the passage 262. The user can then orient the arterial blood collection element 14 and the attached arterial blood module 250 vertically, and depress a plunger rod of the arterial blood collection element 14 so as to vent air bubbles and a clotted sample volume from the arterial blood collection element 14 into a venting chamber 265 within the rotatable stopcock 260. While not shown, it is to be understood that one end of the venting chamber 265 may include a venting plug which will allow air to pass therethrough until wet, at which point the venting plug will seal to prevent the further passage of air and blood.

Next, FIGS. 42-44 illustrate an arterial blood module 300 in accordance with another aspect of the present disclosure. The arterial blood module 300 includes a safety shield assembly 304 coupled to a hub body 302, with hub body 302 having a proximal coupling interface 310 being couplable to, e.g., an arterial blood collection element 14 by way of a threaded luer coupling. The safety shield assembly 304 may include, e.g., a telescopic safety shield and a spring (not shown) for biasing the respective members of the telescopic safety shield toward a deployed position, as shown in FIGS. 42 and 44. Furthermore, a needle 306 is provided in fluid communication with the hub body 302, with the safety shield assembly 304 configured to selectively cover the needle 306.

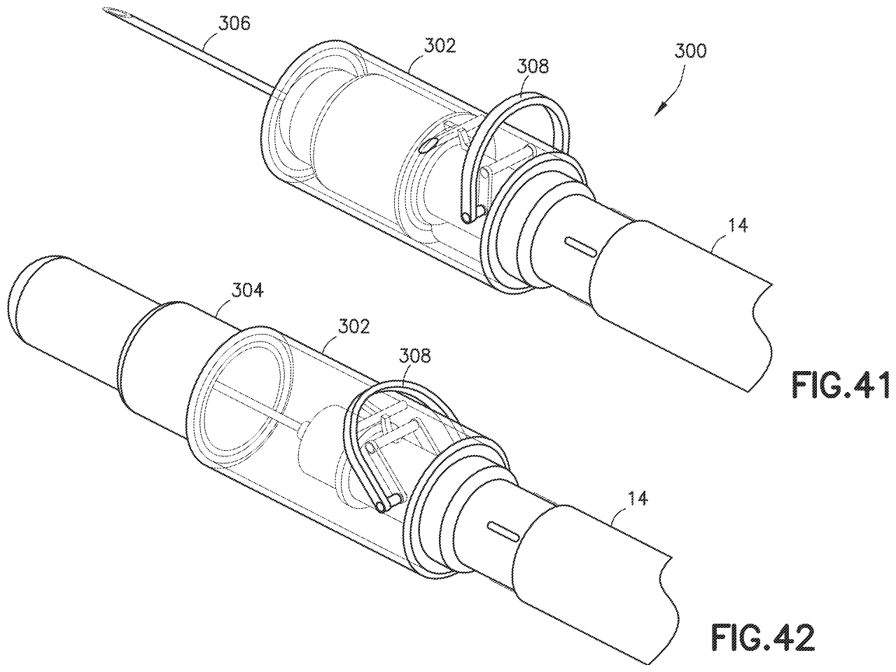

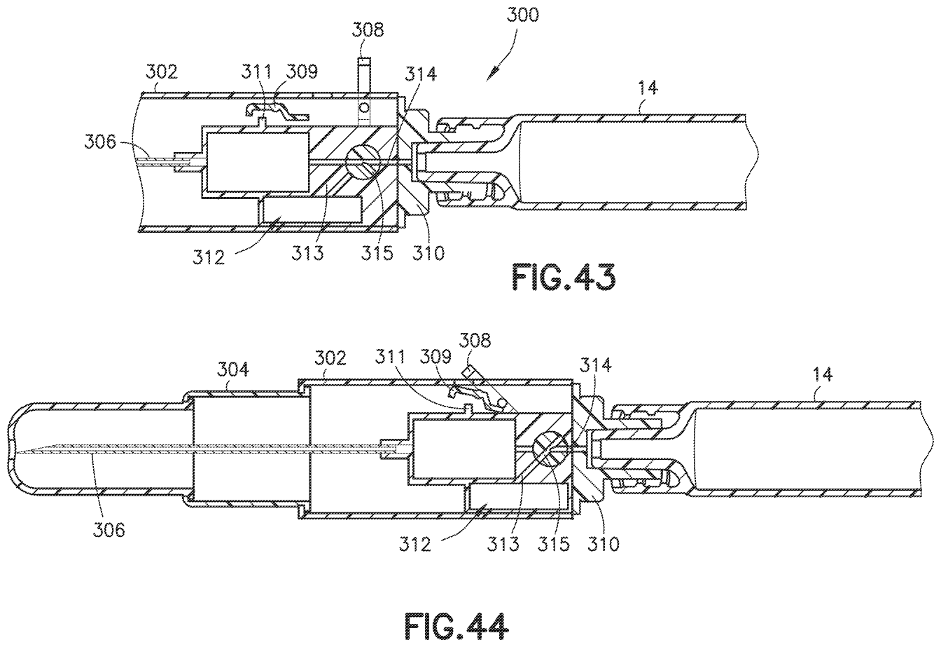

Arterial blood module 300 further includes a rotary valve 315 operably coupled to a lever member 308. When the lever member 308 is in a first position (as shown in FIGS. 41 and 43), the rotary valve 315 is in a first configuration such that the needle 306 and a passage 314 are in fluid communication with one another, thereby allowing for a blood sample to flow from the needle 306 to the arterial blood collection element 14 via the rotary valve 315 and passage 314.

Conversely, when the lever member 308 is in a second position (as shown in FIGS. 42 and 44), the rotary valve 315 is in a second configuration such that the needle 306 and passage 314 are no longer in fluid communication. Rather, in the second configuration, the rotary valve 315 fluidly couples the passage 314 to a conduit 313 leading to a venting chamber 312 within the hub body 302. In such a configuration, the user can depress a plunger rod of the arterial blood collection element 14 so as to vent air bubbles and a clotted sample volume from the arterial blood collection element 14 into a venting chamber 312. While not shown, it is to be understood that one end of the venting chamber 312 may include a venting plug which will allow air to pass therethrough until wet, at which point the venting plug will seal to prevent the further passage of air and blood.

Additionally, arterial blood module 300 may include a latch member 309 pivotally coupled to the hub body 302. The latch member 309 may be configured to selectively hold and release the safety shield assembly 304 based on the position of the lever member 308. That is, when the lever member 308 is in a first position (as shown in FIGS. 41 and 43), the lever member 308 does not contact any portion of the latch member 309, thereby allowing the latch member 309 to hold the safety shield assembly 304 in an undeployed position adjacent a tab portion 311 of the hub body 302 such that the needle 306 remains exposed. However, then the lever member 308 is moved to a second position (as shown in FIGS. 42 and 44), a portion of the lever member 308 contacts the latch member 309, pivoting the latch member 309 in order to release the safety shield assembly 304 to a deployed position, thus substantially covering the needle 306.

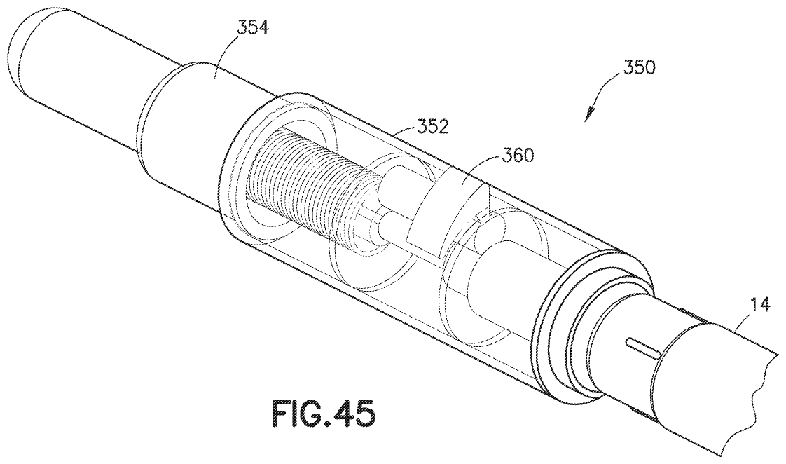

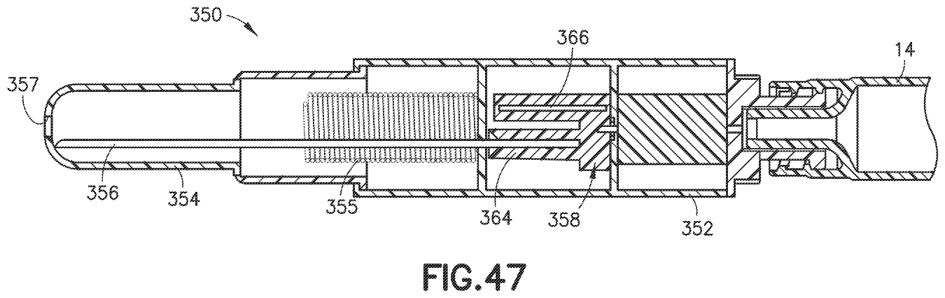

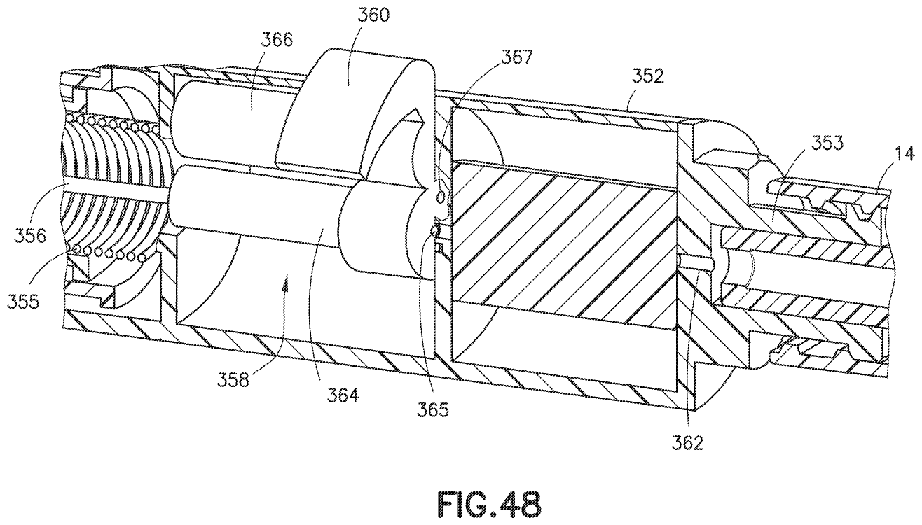

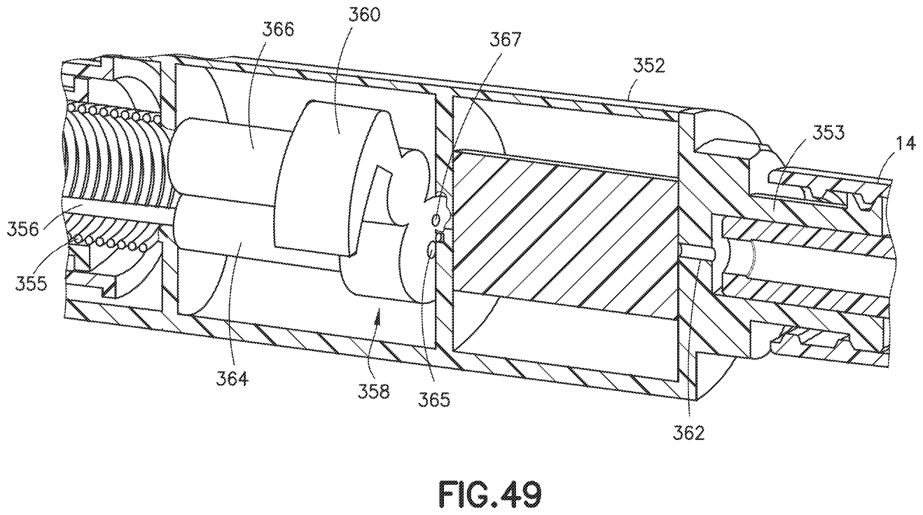

Referring now to FIGS. 45-51, an arterial blood module 350 in accordance with another aspect of the present disclosure. The arterial blood module 350 includes a safety shield assembly 354 coupled to a hub body 352, with hub body 352 having a proximal coupling interface 353 being couplable to, e.g., an arterial blood collection element 14 by way of a threaded luer coupling. The safety shield assembly 354 may include, e.g., a telescopic safety shield and a spring 355 for biasing the respective members of the telescopic safety shield toward a deployed position, as shown in FIGS. 45 and 47. Furthermore, a needle 356 is provided in selective fluid communication with the hub body 352, with the safety shield assembly 354 configured to selectively cover the needle 356.

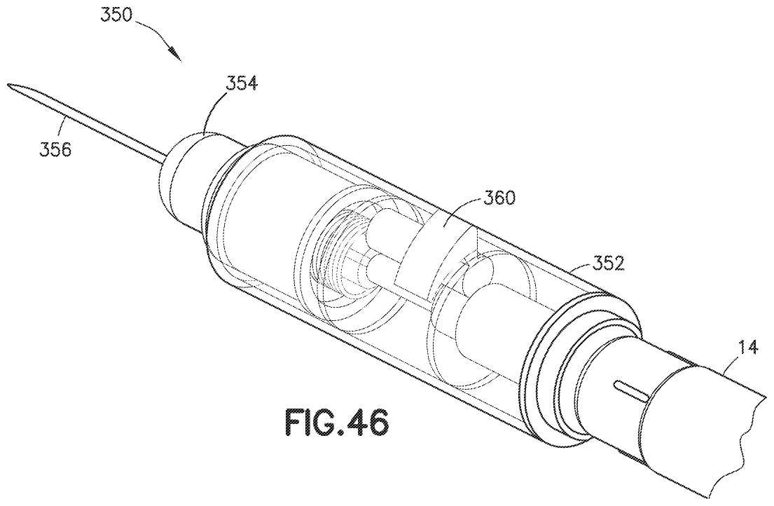

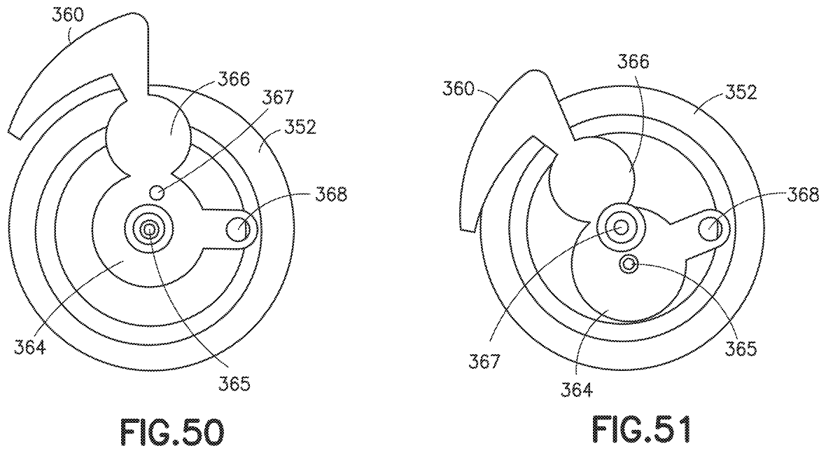

Arterial blood module 350 also includes an eccentric needle carrier 358 having a needle carrying portion 364, a venting chamber portion 366, and a rotatable lever portion 360. As shown in FIGS. 50 and 51, needle carrying portion 364, a venting chamber portion 366, and a rotatable lever portion 360 are configured to eccentrically rotate about a pivot point 368 within the hub body 352. For example, in a first configuration shown in FIGS. 46, 48, and 50, the rotatable lever portion 360 is in a first position such that a first conduit 365 of the needle carrying portion 364 is in fluid communication with a fluid passage 362 leading to the proximal coupling interface 353, thereby enabling a blood sample to pass through the needle 356, the first conduit 365, and the fluid passage 362 into the arterial blood collection element 14.

However, when the rotatable lever portion 360 is moved to a second position, as shown in FIGS. 45, 47, 49, and 51, the needle 356 and first conduit 365 of needle carrying portion 364 are moved out of fluid communication with the fluid passage 362. Instead, a second conduit 367 is moved into fluid communication with the fluid passage, with the second conduit 367 leading to the venting chamber portion 366 of the eccentric needle carrier 360. In such a configuration, the user can depress a plunger rod of the arterial blood collection element 14 so as to vent air bubbles and a clotted sample volume from the arterial blood collection element 14 into a venting chamber of the venting chamber portion 366. While not shown, it is to be understood that one end of the venting chamber portion 366 may include a venting plug which will allow air to pass therethrough until wet, at which point the venting plug will seal to prevent the further passage of air and blood.

As shown in FIG. 47, when the needle carrying portion 364 is rotated to the second position, the needle 354 moves off-center from an opening 357 formed in a distal end of the safety shield assembly 354. Accordingly, rotation of the eccentric needle carrier 358 may also act to lock out retraction of the telescopic needle shield assembly 354, thereby preventing exposure to the needle 356.

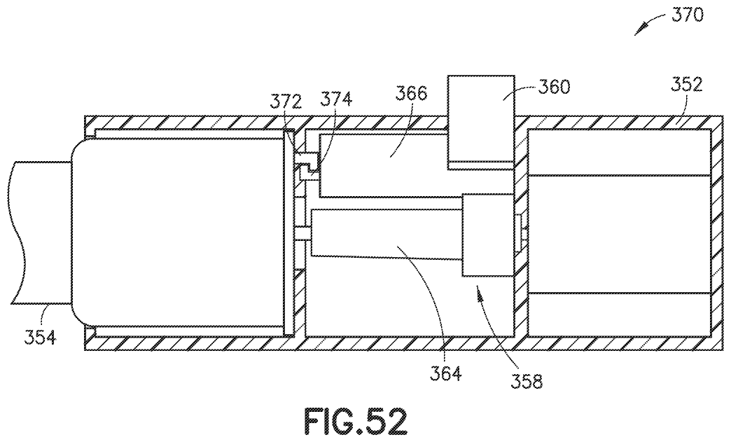

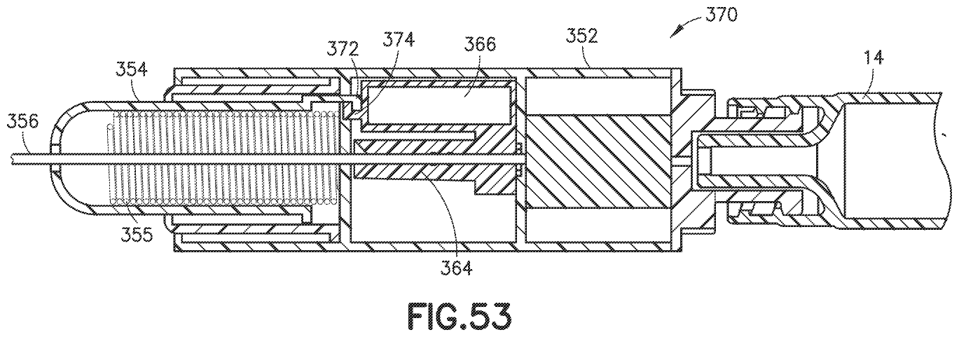

Referring now to FIGS. 52 and 53, an arterial blood module 370 in accordance with another aspect of the present disclosure. Arterial blood module 370 is substantially similar to arterial blood module 350 described above, but incorporates active shielding functionality. Accordingly, the purpose and function of common components between arterial blood module 370 and arterial blood module 350 will not be reiterated herein.

As shown in FIGS. 52 and 53, at least one portion of the safety shield assembly 354 includes a hook member 372 formed at a proximal end thereof, with the hook member 372 size and configured to engage with a corresponding latching member 374 formed on a distal portion of the venting chamber portion 366 of eccentric needle carrier 358. Accordingly, when the eccentric needle carrier 358 is in the first position (as shown in FIGS. 52 and 53), the safety shield assembly 354 is held in an undeployed position such that the needle 356 is exposed. However, when the eccentric needle carrier 358 is rotated to the second position (not shown), the latching member 374 will disengage from the hook member 372, thereby releasing the safety shield assembly 354 such that the safety shield assembly 354 deploys to substantially cover the needle 356. With this configuration, rotation of the eccentric needle carrier 358 may act to simultaneously decouple the needle 356 from fluid communication with the arterial blood collection element 14 and deploy the safety shield assembly 354.

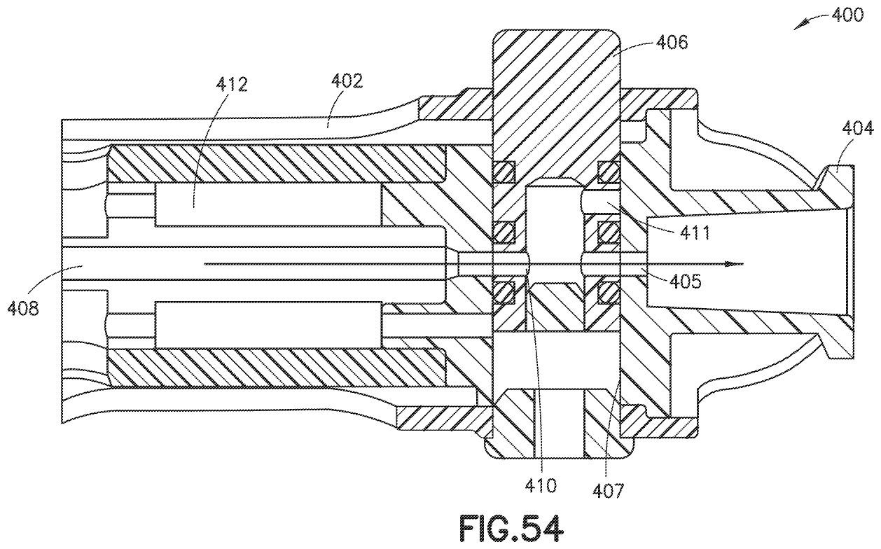

Next, referring to FIGS. 54-57, an arterial blood module 400 in accordance with another aspect of the present disclosure is illustrated. Arterial blood module 400 is configured to move from a blood draw configuration to a venting configuration via a simple push-button actuation.

Arterial blood module 400 includes a hub body 402 having a proximal coupling interface 404, with the proximal coupling interface 404 being configured to removably couple the arterial blood module 400 to an arterial blood collection element such as, e.g., arterial blood collection element 14 described above. While not shown, it is to be understood that arterial blood module 400 may also include a safety shield which may be selectively deployed by a user to cover a needle.

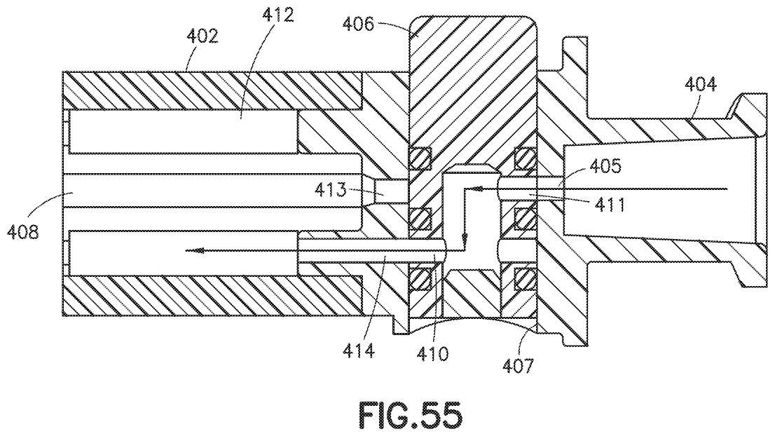

Referring to FIG. 54, arterial blood module 400 is shown with a push-button-style actuator 406 in a first position. The actuator 406 is configured to be slidable within a bore 407 of a housing 420, with the actuator 406 having a first passage 410 and a second passage 411 offset but in fluid communication with one another, as shown in FIGS. 54 and 55. In the first position, the actuator 406 is positioned such that the first passage 410 is aligned with a first opening 413 and a central conduit 408 of the hub body 402, with central conduit 408 configured to be in fluid communication with a needle (not shown) of the arterial blood module 400. The first passage 410 is also with a second opening 405 formed adjacent to the proximal coupling interface 404. Thus, in this first position, a blood sample can pass from the needle, through the actuator 406, and to an arterial blood collection element coupled to the proximal coupling interface 404.

Conversely, referring to FIG. 55, when the actuator 406 is depressed and moved into a second position, the first opening 413 is blocked, thereby blocking fluid communication with the central conduit 408 (and, thus, the needle). Instead, the second passage 411 of actuator 406 is aligned with the second opening 405, while the first passage 410 is placed in fluid communication with a third opening 414 leading to a venting chamber 412 formed within the hub body 402. Accordingly, in this second position, a user can depress a plunger rod of the arterial blood collection element (not shown) so as to vent air bubbles and a clotted sample volume from the arterial blood collection element into the venting chamber 412. While not shown, it is to be understood that one end of the venting chamber 412 may include a venting plug which will allow air to pass therethrough until wet, at which point the venting plug will seal to prevent the further passage of air and blood.

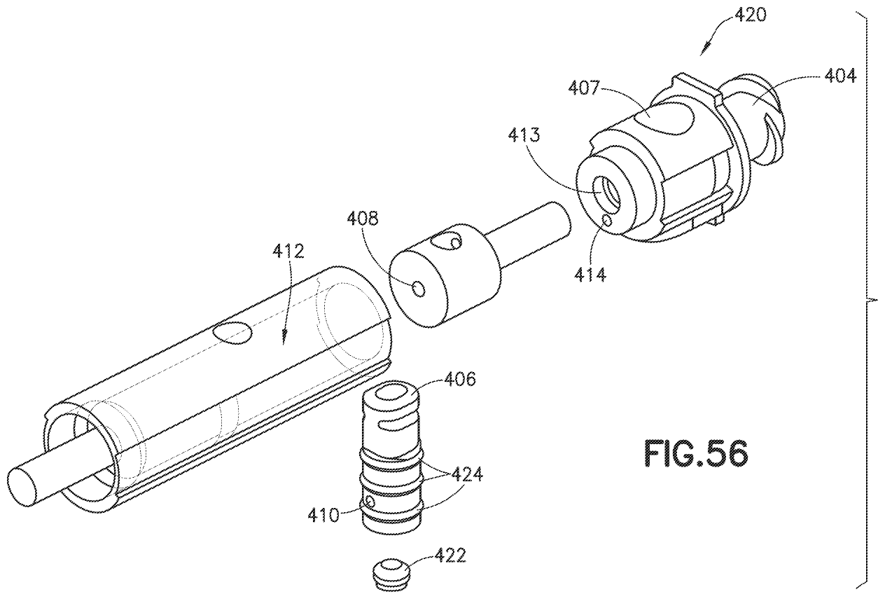

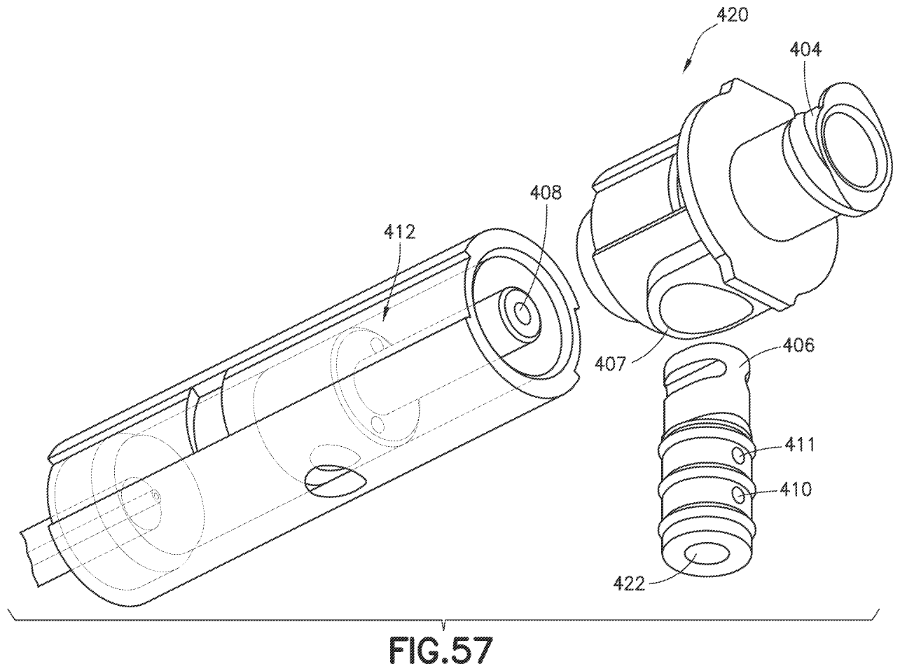

As shown in FIGS. 56 and 57, the actuator 406 may incorporate a plurality of O-ring-style seals 424 so as to avoid leakage of air and/or blood form the bore 407. The seals 424 may be formed of any appropriate material such as, e.g., TPE. Furthermore, a plug 422 may be provided in one end of the actuator 406 so as to enable simplified molding of the actuator 406 having interconnected passages formed therethrough.

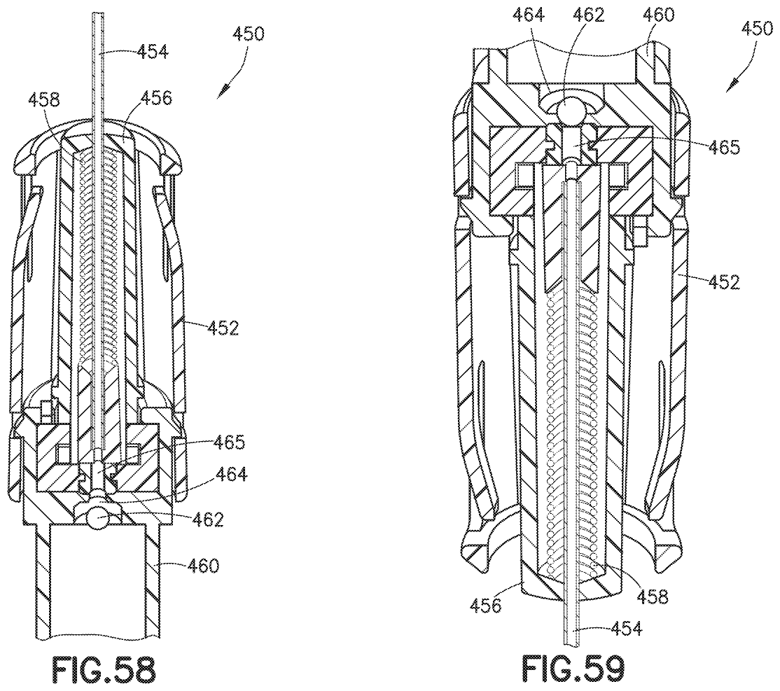

Next, referring to FIGS. 58 and 59, a gravity-based “lock valve” configured to prevent blood from flowing into a chamber when an arterial blood module 450 is pointed downward (i.e., a needle 454 is pointed downward) is illustrated. As shown, arterial blood module 450 includes a housing 452, a needle 454, and a safety shield 456 selectively deployable by a spring 458. A base 460 is also provided, with base 460 configured to allow fluid communication between the needle 454 and an arterial blood collection element coupled to the arterial blood module 450.

Arterial blood module 450 also includes a moving ball 462 movably disposed between the base 460 and a passage 465 fluidly coupled to the needle 454. As shown in FIG. 58, when the arterial blood module 450 is held in an upright position (i.e., the needle 454 is pointed upward), the ball 462 naturally moves away from a seat 464 adjacent the passage 465, thereby allowing fluid to flow from the base 460 to the needle 454. However, referring to FIG. 59, when the arterial blood module 450 is inverted so as to be held in a downward position, gravitational forces direct the ball 462 into the seat 464, thereby blocking the passage of blood into the passage 465 when the device is actuated with the needle 454 pointing downward. Regardless of the position of the needle 454, though, the safety shield 456 may be deployed to substantially cover the needle 454.

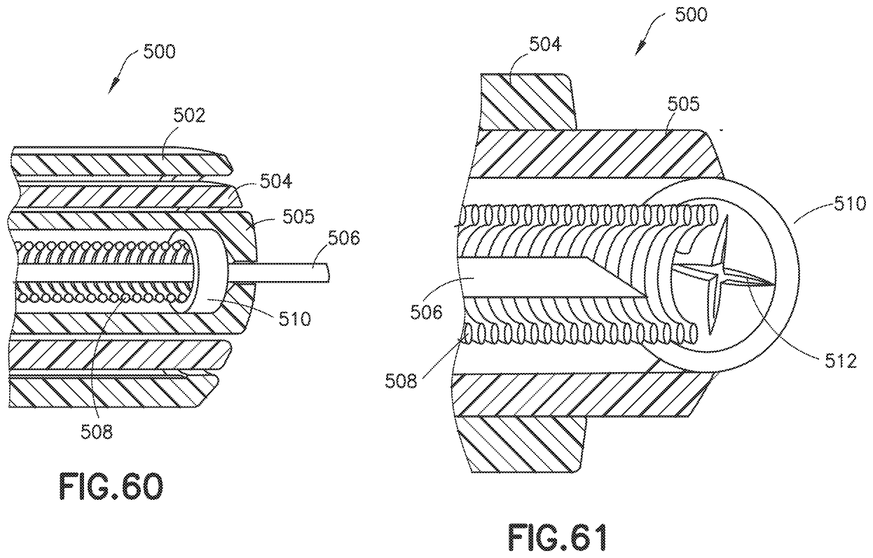

Referring now to FIGS. 60 and 61, a safety shield assembly 500 in accordance with another aspect of the present disclosure is illustrated. Safety shield assembly 500 includes a telescopic needle shield comprising at least an outer telescopic member 502, a middle telescopic member 504, and an inner telescopic member 505. When selectively biased by a spring 508, the outer telescopic member 502, middle telescopic member 504, and inner telescopic member 505 act to substantially surround a needle 506 coupled to, e.g., an arterial blood module.

The safety shield assembly 500 also includes an insert 510 disposed at or near distal end of an interior portion of the inner telescopic member 505. The insert 510 includes a slot 512, as shown in FIG. 61. The slot 512 may be configured to allow needle 506 to pass therethrough in a first configuration (as shown in FIG. 60), but then substantially close when the needle 506 is surrounded by the safety shield assembly (as shown in FIG. 61). The insert 510 may be formed of any appropriate material, such as an elastomeric or foam material.

When the safety shield is actuated to close, the insert 510 moves along the needle 506, wiping the outer surface of the needle 506 as the inner telescopic member 505 travels to its terminus. Then, once beyond the distal tip of the needle 506, the slot 512 of needle 510 substantially closes, thereby preventing blood splatter out of the safety shield assembly 500.

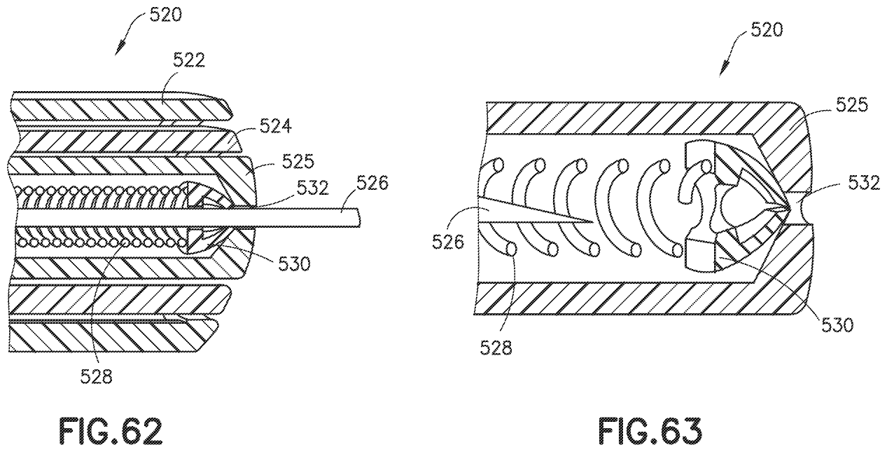

Next, referring to FIGS. 62 and 63, a safety shield assembly 520 in accordance with another aspect of the present disclosure is illustrated. Similar to safety shield assembly 500 described above with respect to FIGS. 60 and 61, safety shield assembly 520 includes a telescopic needle shield comprising at least an outer telescopic member 522, a middle telescopic member 524, and an inner telescopic member 525. When selectively biased by a spring 528, the outer telescopic member 522, middle telescopic member 524, and inner telescopic member 525 act to substantially surround a needle 526 coupled to, e.g., an arterial blood module.

The safety shield assembly 520 also includes an insert 530 disposed at or near distal end of an interior portion of the inner telescopic member 525. The insert 530 may include a living hinge, allowing the insert 530 to pinch or close when pressed against an interior distal end of the inner telescopic member 525, thereby substantially sealing a distal opening 532 of the inner telescopic member 525 when the safety shield assembly 520 is fully deployed. The insert 530 may be configured to allow needle 526 to pass therethrough in a first configuration (as shown in FIG. 62), but then substantially close when the needle 526 is substantially surrounded by the safety shield assembly (as shown in FIG. 63). When the safety shield is actuated to close, the insert 530 moves along the needle 526, wiping the outer surface of the needle 526 as the inner telescopic member 525 travels to its terminus. Then, once beyond the distal tip of the needle 526, force applied by the spring 528 on the insert 530 causes the insert to pivot about the living hinge to substantially close at a distal tip thereof, thereby preventing blood splatter out of the safety shield assembly 520.

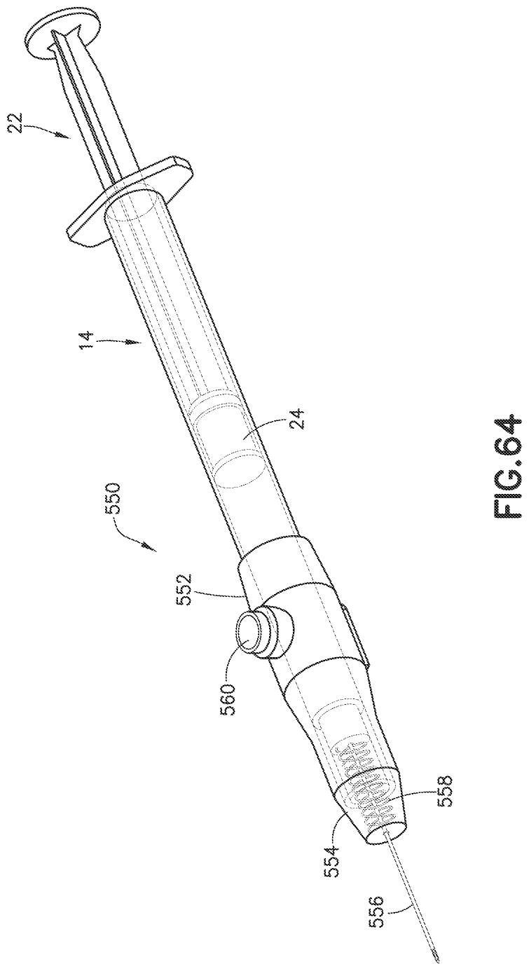

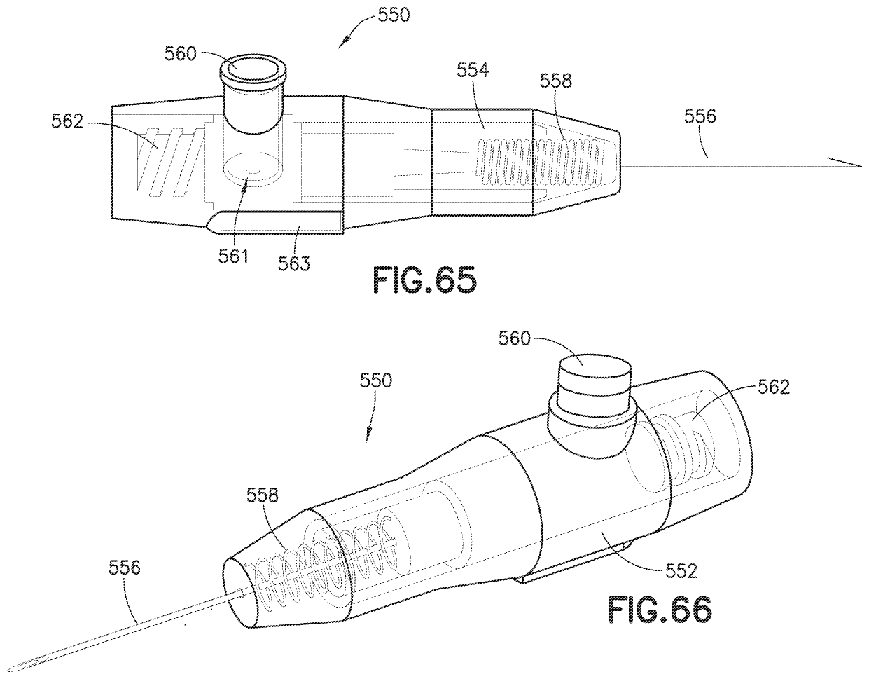

Referring now to FIGS. 64-69, an arterial blood module 550 in accordance with another aspect of the present disclosure is illustrated. As with the various arterial blood modules described above, the arterial blood module 550 is configured to be coupled to, e.g., an arterial blood collection element 14 having a plunger 22 and stopper 24 for the collection and dispensation of a blood sample.

The arterial blood module 550 includes a hub body 552 having a proximal coupling interface 562, with the proximal coupling interface 562 being configured to removably couple the arterial blood module 550 to an arterial blood collection element such as, e.g., arterial blood collection element 14 described above. Arterial blood module 550 also includes safety shield 554, which may be selectively deployed by a user to cover a needle 556. As will be described in further detail below, deployment of the safety shield 554 is initiated by an actuator 560 housed within the hub body 552.

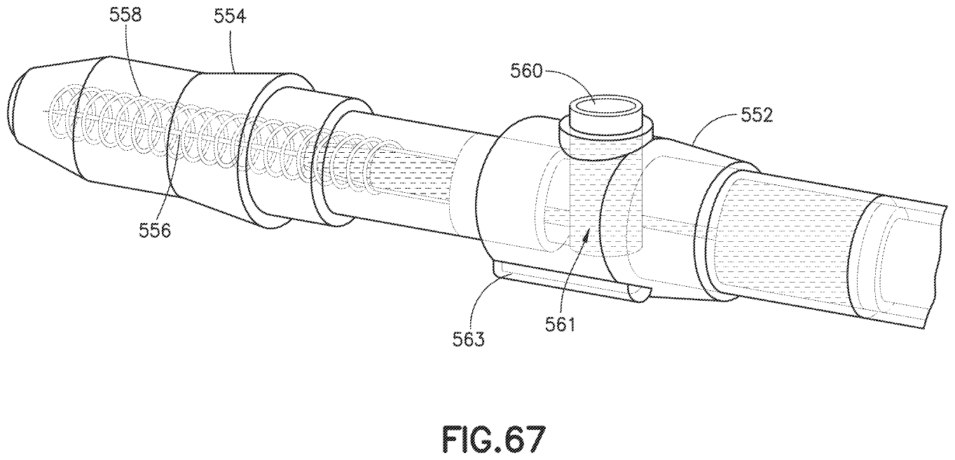

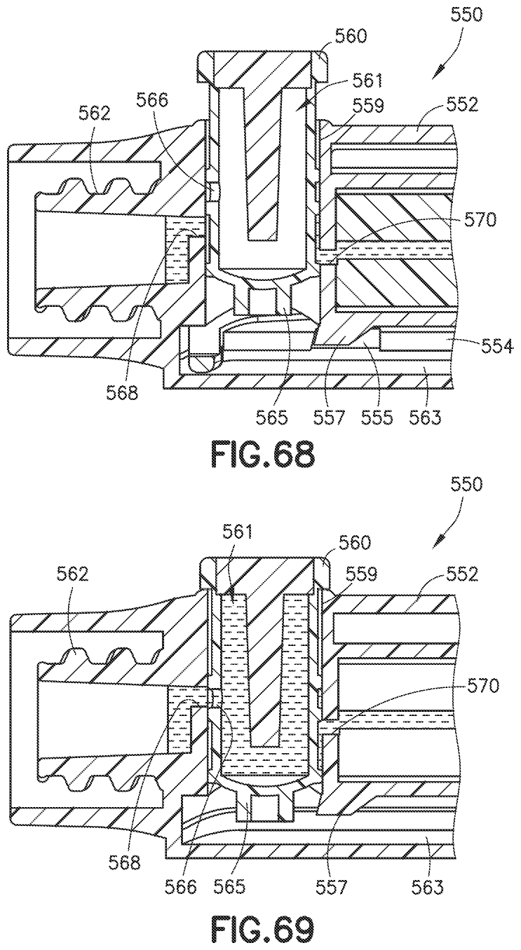

FIGS. 64-66 illustrate the arterial blood module 550 in a first configuration, where the safety shield 554 is in an undeployed position such that the needle 556 is at least partially exposed. In this first configuration, the safety shield 554 may be releasably secured to the hub body 552 via an interface between a protruding portion 557 of the hub body 552 and a detent portion 555 of the safety shield 554, as is shown in FIG. 68. The proximal portion of safety shield 554 including the detent portion 555 is configured to be received within a slot 563 of the hub body 552. The slot 563 is in communication with an actuator bore 559 formed within the hub body 552, with the actuator 560 being slidable within the actuator bore 559 in a direction substantially transverse to the direction of slot 563 and, thus, substantially transverse to the direction of deployment of the safety shield 554. Accordingly, when the actuator 560 is depressed, a base portion 565 of actuator 560 contacts the proximal portion of the safety shield 554, disengaging the detent portion 555 of the safety shield 554 from the protruding portion 557 of the hub body 552 such that the safety shield 554 is telescopically deployed over the needle 556 by way of spring force applied by a spring 558, as is shown in FIG. 67.

Referring again to FIG. 68, when the safety shield 554 is in an undeployed position (i.e., with the needle 556 is exposed), the arterial blood module 550 is configured to provide a fluid path between the needle 556 and an opening 568 in communication with the proximal coupling interface 562. More specifically, the arterial blood module 550 includes a flow channel 570 passing through the hub body 552, wherein the flow channel 570 is in fluid communication with the needle 556. While not shown, the flow channel 570 may be in communication with, e.g., a sample stabilizer within a portion of the hub body 552. In this configuration, a blood sample may flow through the needle 556, the flow channel 570, and around the actuator 560 to the opening 568, where the sample may be drawn into, e.g., an arterial blood collection element coupled to the proximal coupling interface 562. Radial seals provided circumferentially around the actuator 560 may act to provide a fluid-tight channel between the flow channel 570 and opening 568.

Conversely, referring to FIG. 69, when the actuator 560 is depressed to deploy the safety shield 554, the fluid pathway between the opening 568 and the flow channel 570 is also disconnected, thereby stopping fluid flow between the needle 556 and the opening 568. More specifically, depression of the actuator 560 within the actuator bore 559 aligns an orifice 566 formed in the actuator 560 with the opening 568. The orifice 566 is in fluid communication with a vent chamber 561 formed within the actuator 560. The actuator 560 may also include a venting plug in communication with the vent chamber 561 that allows air to pass therethrough, while preventing a blood sample from passing therethrough. In some embodiments, the venting plug may include a hydrophobic filter. In this manner, any air bubbles contained within an arterial blood collection element coupled to the arterial blood module 550 can be expelled outside of the system through the vent chamber 561 and out the venting plug formed as part of the actuator 560.

While not specifically shown, it is to be understood that hub body 552 and safety shield 554 may be configured such that safety shield 554 is substantially locked in place upon deployment, thereby preventing unwanted or unintended exposure of the needle 556. The entire arterial blood module 550 may be decoupled from arterial blood collection element 14 after a blood draw and air bubble venting procedure, and the entire arterial blood module 550 may be disposed of in an appropriate sharps container.

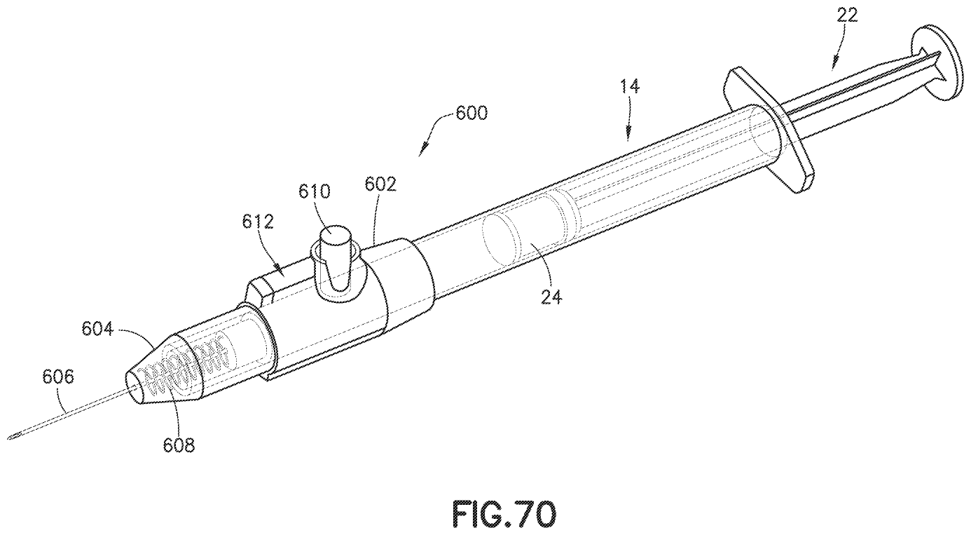

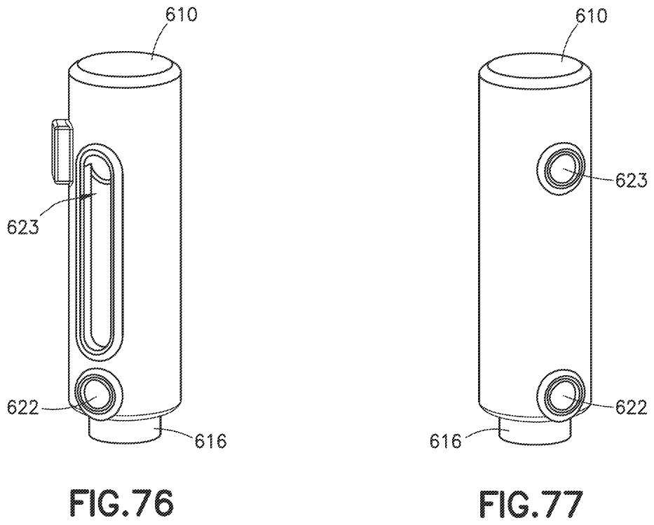

Next, referring to FIGS. 70-77, various aspects of an arterial blood module 600 in accordance with another aspect of the present disclosure is illustrated. As with the arterial blood modules described above, the arterial blood module 600 is configured to be coupled to, e.g., an arterial blood collection element 14 having a plunger 22 and stopper 24 for the collection and dispensation of a blood sample, as is shown in FIG. 70.

The arterial blood module 600 includes a hub body 602 having a proximal coupling interface 611, with the proximal coupling interface 611 being configured to removably couple the arterial blood module 600 to an arterial blood collection element such as, e.g., arterial blood collection element 14 described above. Arterial blood module 600 also includes safety shield 604, which may be selectively deployed by a user to cover a needle 606. As will be described in further detail below, deployment of the safety shield 604 is initiated by an actuator 610 housed within the hub body 602.

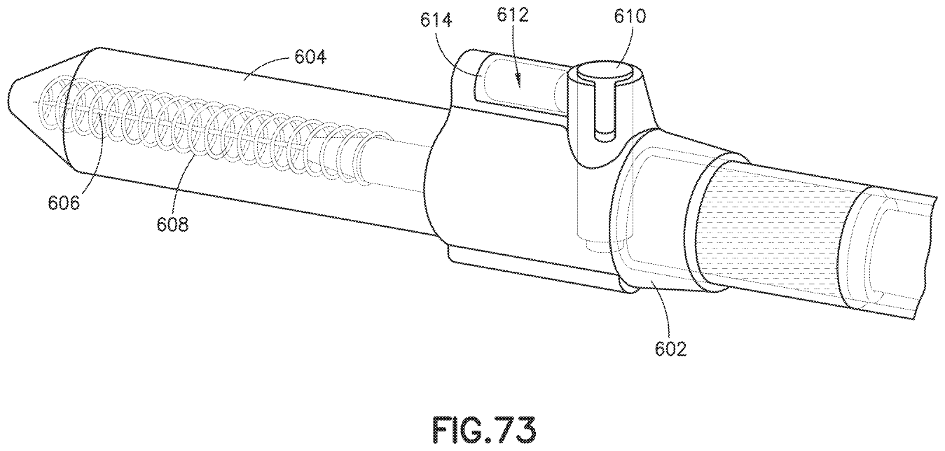

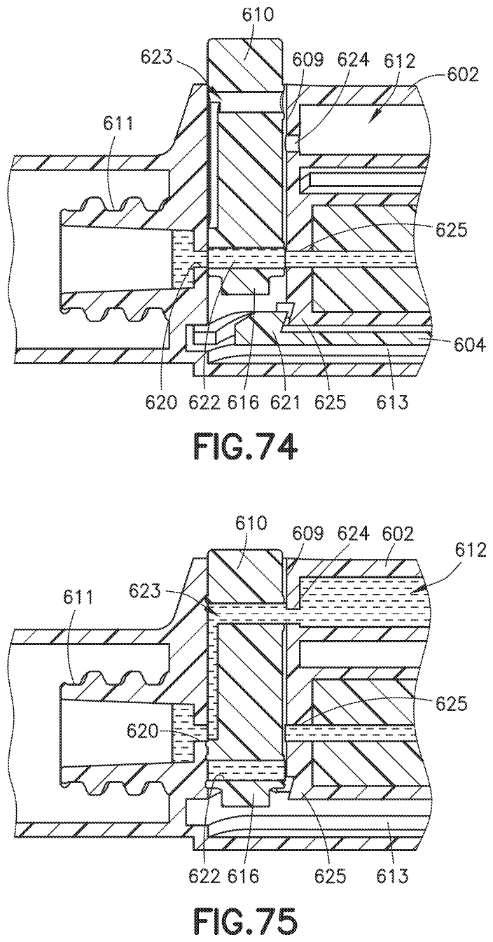

FIGS. 70-72 illustrate the arterial blood module 600 in a first configuration, where the safety shield 604 is in an undeployed position such that the needle 606 is at least partially exposed. In this first configuration, the safety shield 604 may be releasably secured to the hub body 602 via an interface between a protruding portion 625 of the hub body 602 and a detent portion 621 of the safety shield 604, as is shown in FIG. 74. The proximal portion of safety shield 604 including the detent portion 621 is configured to be received within a slot 613 of the hub body 602. The slot 613 is in communication with an actuator bore 609 formed within the hub body 602, with the actuator 610 being slidable within the actuator bore 609 in a direction substantially transverse to the direction of slot 613 and, thus, substantially transverse to the direction of deployment of the safety shield 604. Accordingly, when the actuator 610 is depressed, a base portion 616 of actuator 610 contacts the proximal portion of the safety shield 604, disengaging the detent portion 621 of the safety shield 604 from the protruding portion 625 of the hub body 602 such that the safety shield 604 is telescopically deployed over the needle 606 by way of spring force applied by a spring 608, as is shown in FIG. 73.

As shown in FIG. 74, when the safety shield 604 is in an undeployed position (i.e., with the needle 606 is exposed), the arterial blood module 600 is configured to provide a fluid path between the needle 606 and an opening 620 in communication with the proximal coupling interface 611. More specifically, the arterial blood module 600 includes a flow channel 625 passing through the hub body 602, wherein the flow channel 625 is in fluid communication with the needle 606. While not shown, the flow channel 625 may be in communication with, e.g., a sample stabilizer within a portion of the hub body 602. In this configuration, a blood sample may flow through the needle 606, through the flow channel 625, through a first channel 622 formed through the actuator 610, and through the opening 620, where the sample may be drawn into, e.g., an arterial blood collection element coupled to the proximal coupling interface 611.

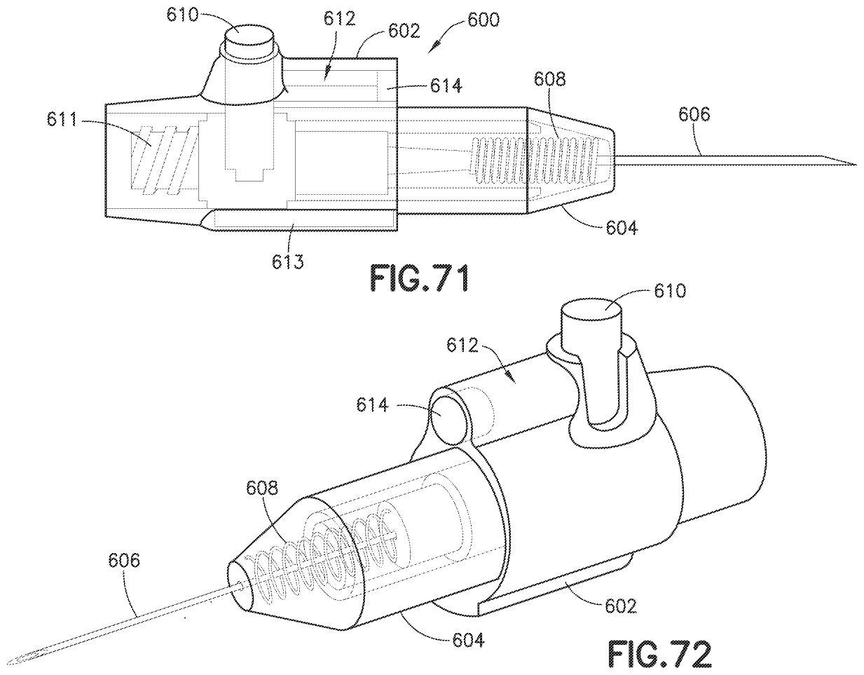

However, referring to FIG. 75, when the actuator 610 is depressed to deploy the safety shield 604, the first channel 622 is fluidly decoupled from the opening 620 and the flow channel 625, thereby preventing fluid flow between the needle 606 and the opening 620. Instead, depression of the actuator 610 within the actuator bore 609 aligns a second, substantially L-shaped channel 623 formed in the actuator 560 with the opening 620. The second channel 623 is in fluid communication with a secondary opening 624, which is fluidly coupled to a vent chamber 612 formed within the hub body 602. As shown in FIGS. 71-73, a venting plug 614 is provided in communication with the vent chamber 612, which allows air to pass therethrough until fully wetted, while preventing a blood sample from passing therethrough. In some embodiments, the venting plug may 614 include a hydrophobic filter. In this manner, any air bubbles contained within an arterial blood collection element coupled to the arterial blood module 600 can be expelled outside of the system through the vent chamber 612 formed integrally within the hub body 602 and out the venting plug 614.

While not specifically shown, it is to be understood that hub body 602 and safety shield 604 may be configured such that safety shield 604 is substantially locked in place upon deployment, thereby preventing unwanted or unintended exposure of the needle 606. The entire arterial blood module 600 may be decoupled from arterial blood collection element 14 after a blood draw and air bubble venting procedure, and the entire arterial blood module 600 may be disposed of in an appropriate sharps container.

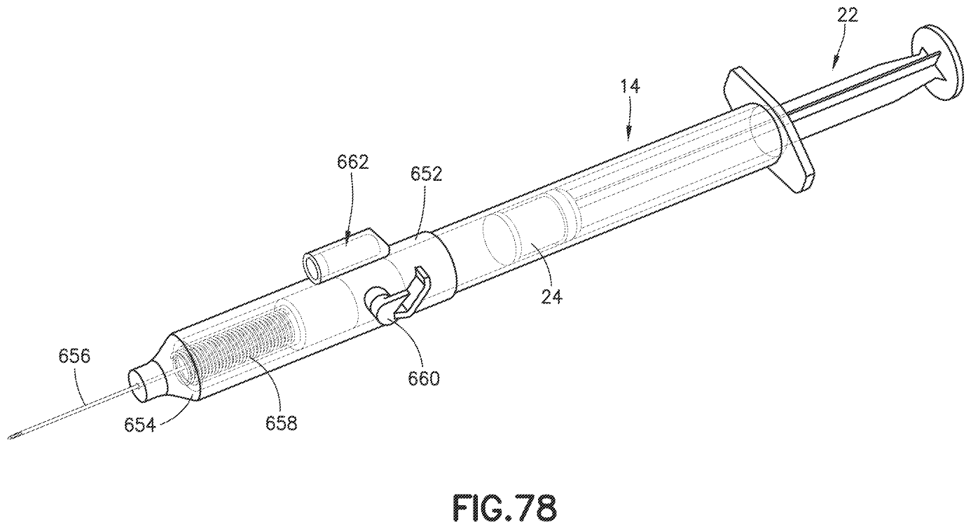

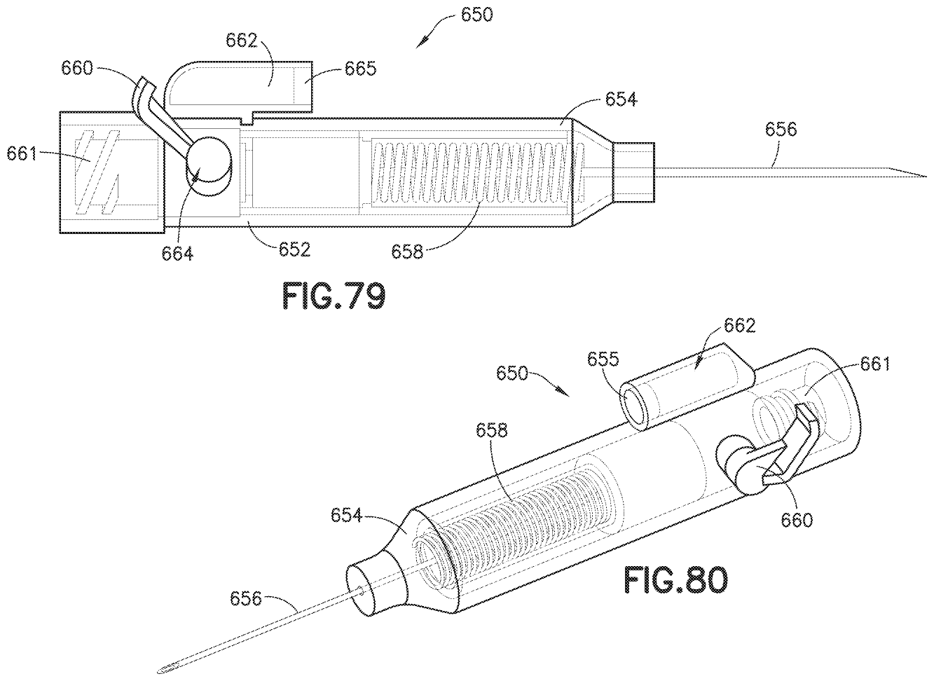

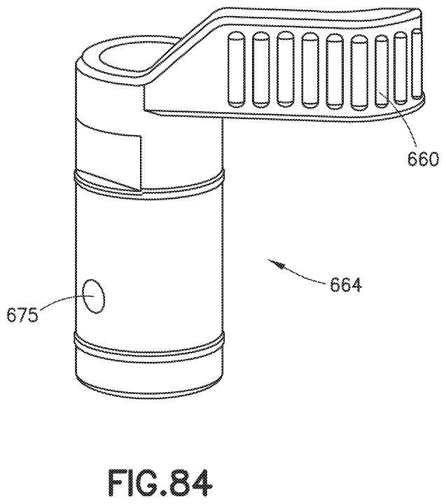

Referring now to FIGS. 78-84, an arterial blood module 650 in accordance with another aspect of the present disclosure is illustrated. As with the arterial blood modules described above, the arterial blood module 650 is configured to be coupled to, e.g., an arterial blood collection element 14 having a plunger 22 and stopper 24 for the collection and dispensation of a blood sample, as is shown in FIG. 78.

The arterial blood module 650 includes a hub body 652 having a proximal coupling interface 661, with the proximal coupling interface 661 being configured to removably couple the arterial blood module 650 to an arterial blood collection element such as, e.g., arterial blood collection element 14 described above. Arterial blood module 650 also includes safety shield 654, which may be selectively deployed by a user to cover a needle 656. As will be described in further detail below, deployment of the safety shield 654 is initiated by rotation of a stopcock valve 664 at least partially housed within the hub body 652.

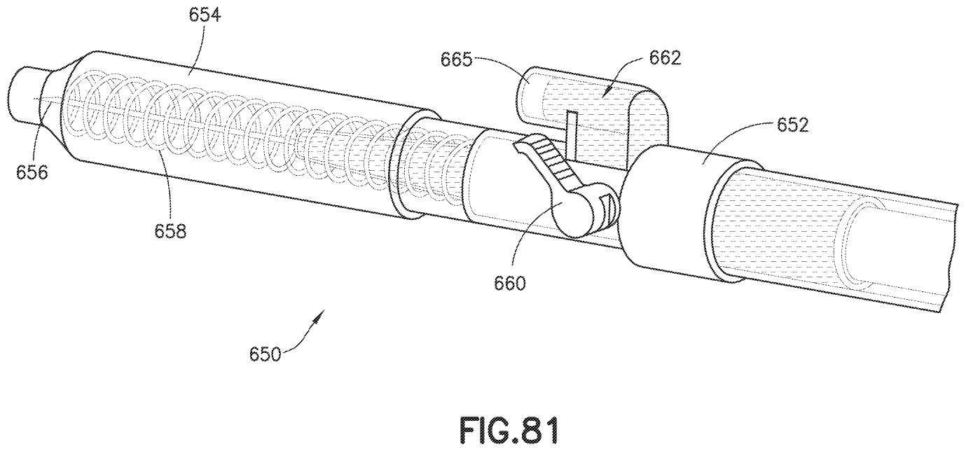

FIGS. 78-80 illustrate the arterial blood module 650 in a first configuration, where the safety shield 654 is in an undeployed position such that the needle 656 is at least partially exposed. In this first configuration, the safety shield 654 may be releasably secured to the hub body 652 via an interface between at least one of a lever actuator 660 and the stopcock valve 664 to which the lever actuator 660 is coupled. When the lever actuator 660 is actuated such that the stopcock valve 664 is rotated, the safety shield 654 may also be released to telescopically deploy the safety shield 654 over the needle 656 by way of spring force applied by a spring 658, as is shown in FIG. 81.

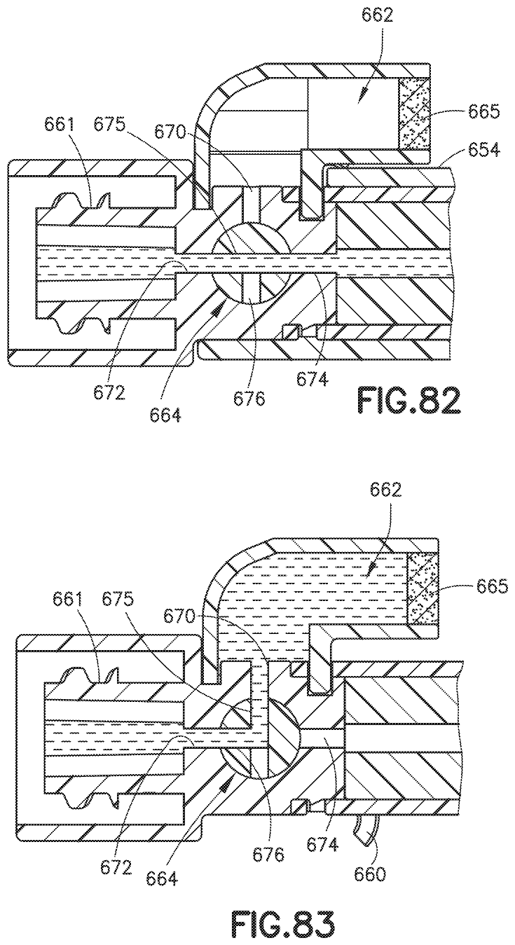

Referring to FIG. 82, when the safety shield 654 is in an undeployed position (i.e., with the needle 656 is exposed), the arterial blood module 650 is configured to provide a fluid path between the needle 656 and an opening 672 in communication with the proximal coupling interface 661. More specifically, the arterial blood module 650 includes a flow channel 674 passing through the hub body 652, wherein the flow channel 674 is in fluid communication with the needle 656. While not shown, the flow channel 674 may be in communication with, e.g., a sample stabilizer within a portion of the hub body 652. In this configuration, the lever actuator 660 and stopcock valve 664 are in a first position such that a blood sample may flow through the needle 656, through the flow channel 674, through a first channel 675 formed through the stopcock valve 664, and through the opening 672, where the sample may be drawn into, e.g., an arterial blood collection element coupled to the proximal coupling interface 661.

Conversely, referring to FIG. 83, when the lever actuator 660 is pressed to provide, e.g., a 90° rotation of the stopcock valve 664, not only may the safety shield 654 be deployed, but the first channel 675 is fluidly decoupled from the flow channel 674, thereby preventing fluid flow between the needle 656 and the opening 672. Instead, rotation of the stopcock valve 664 aligns a secondary channel 676 formed in the stopcock valve 664 with the opening 672, while also aligning the first channel 675 with a secondary opening 670. The secondary opening 670 is fluidly coupled to a vent chamber 662 formed within the hub body 652. As shown in 79-86, a venting plug 665 is provided in communication with the vent chamber 662, which allows air to pass therethrough until fully wetted, while preventing a blood sample from passing therethrough. In some embodiments, the venting plug may 665 include a hydrophobic filter. In this manner, any air bubbles contained within an arterial blood collection element coupled to the arterial blood module 650 can be expelled outside of the system through the vent chamber 662 formed integrally within the hub body 602 and out the venting plug 614, and the vent chamber 662 can hold a small volume of clotted blood sample therein.

While not specifically shown, it is to be understood that hub body 652 and safety shield 654 may be configured such that safety shield 654 is substantially locked in place upon deployment, thereby preventing unwanted or unintended exposure of the needle 656. The entire arterial blood module 650 may be decoupled from arterial blood collection element 14 after a blood draw and air bubble venting procedure, and the entire arterial blood module 650 may be disposed of in an appropriate sharps container.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. An arterial blood collection system, comprising:
an arterial blood collection element defining a collection chamber; and
an arterial blood module removably connectable to a portion of the arterial blood collection element, the arterial blood module comprising:
a hub body;
a needle extending from the hub body;
a safety shield engaged with a portion of the hub body and transitionable from a first shield position in which a portion of the needle is exposed to a second shield position in which the needle is shielded by a portion of the safety shield; and
a venting chamber, wherein the venting chamber is in fluid communication with the hub body to vent air bubbles from a collected blood sample within the collection chamber.

2. The arterial blood collection system of claim 1, wherein the arterial blood module further comprises an actuator transitionable from a first actuator position to a second actuator position, wherein the second actuator position releases the safety shield to the second shield position.

3. The arterial blood collection system of claim 2, wherein the actuator comprises a push button.

4. The arterial blood collection system of claim 2, wherein the safety shield comprises telescoping shields.

5. The arterial blood collection system of claim 2, wherein the venting chamber is formed within the actuator.

6. The arterial blood collection system of claim 1, wherein the hub body of the arterial blood module defines the venting chamber.

7. The arterial blood collection system of claim 1, wherein the arterial blood module further comprises a venting plug that allows air to pass therethrough and prevents a blood sample from passing therethrough, wherein a portion of the venting plug is in communication with the venting chamber.

8. The arterial blood collection system of claim 1, wherein the needle and safety shield are selectively removable from the hub body.

9. The arterial blood collection system of claim 8, wherein the needle and safety shield are removable from the hub body only when the safety shield is in the second shield position.

10. The arterial blood collection system of claim 1, wherein the actuator comprises an actuator lever and stopcock valve.

11. The arterial blood collection system of claim 1, wherein the arterial blood collection module further comprises an eccentric needle carrier, the eccentric needle carrier comprising a needle carrying portion coupled to and in fluid communication with the needle, a venting chamber portion, and a rotatable lever portion.

12. The arterial blood collection system of claim 1, further comprising an insert positioned at a distal end of an interior portion of the safety shield to seal an opening in the safety shield when in the second shield position in which the needle is shielded by a portion of the safety shield.

13. An arterial blood module, comprising:
a hub body;
a needle extending from the hub body;
a safety shield engaged with a portion of the hub body and transitionable from a first shield position in which a portion of the needle is exposed to a second shield position in which the needle is shielded by a portion of the safety shield; and
a venting chamber, wherein the venting chamber is in fluid communication with the hub body to vent air bubbles from a collected blood sample within the collection chamber.

14. The arterial blood module of claim 13, wherein the arterial blood module is removably connectable to a portion of an arterial blood collection element.

15. The arterial blood module of claim 14, wherein the arterial blood module is connectable to a portion of the arterial blood collection element by way of a threaded luer connection.

16. The arterial blood module of claim 13, further comprising an actuator transitionable from a first actuator position to a second actuator position, wherein the second actuator position releases the safety shield to the second shield position.

17. The arterial blood module of claim 16, wherein the venting chamber is formed within the actuator.

18. The arterial blood module of claim 16, wherein the actuator comprises a push button.

19. The arterial blood module of claim 16, wherein the actuator comprises an actuator lever and stopcock valve.

20. The arterial blood module of claim 13, wherein the hub body of the arterial blood module defines the venting chamber.

21. The arterial blood module of claim 13, further comprising venting plug that allows air to pass therethrough and prevents a blood sample from passing therethrough, wherein a portion of the venting plug is in communication with the venting chamber.
